(19) United States
(12) Patent Application Publication
(10) Pub. No.: US 2023/0233567 A1
(43) Pub. Date: Jul. 27, 2023

(54) BELVARAFENIB FOR USE IN CANCER TREATMENT

(71) Applicants: Hanmi Pharm. Co., Ltd., Seoul (KR); Genentech, Inc., South San Francisco, CA (US)

(72) Inventors: Tae Won KIM, Seoul (KR); Yoon-hee HONG, Seoul (KR); Young Su NOH, Seoul (KR); Matthew Tsn-Wei CHANG, Alamo, CA (US); Shiva MALEK, Burlingame, CA (US); Yibing YAN, San Francisco, CA (US); Ivana Yen Yen YEN, Millbrae, CA (US)

(21) Appl. No.: 17/999,799

(22) PCT Filed: May 17, 2021
(86) PCT No.: PCT/US2021/032667

§ 371 Date: Nov. 23, 2022

Related U.S. Application Data
(60) Provisional application No. 63/030,171, filed on May 26, 2020.

(51) Int. Cl. A61K 31/519; A61P 35/00; A61P 17/00

(52) U.S. Cl. CPC A61K 31/519; A61P 35/00; A61P 17/00

(57) ABSTRACT

Provided are methods for the use of belvarafenib to treat cancer having at least one mutation selected from a BRAF^{V600E} mutation, a KRAS^{G12V} mutation, a KRAS^{G12D} mutation, a KRAS^{G12C} mutation, a KRAS^{G12R} mutation, a KRAS^{G13D} mutation, a KRAS^{Q61H} mutation, a NRAS^{G12D} mutation, a NRAS^{G13D} mutation, a NRAS^{Q61K} mutation, a NRAS^{Q61L} mutation, a NRAS^{Q61R} mutation, and a NRAS^{G12C} mutation.

### CROSS-REFERENCE TO RELATED APPLICATION

This application claims priority benefit of U.S. Provisional Application Ser. No. 63/030,171 filed on May 26, 2020, which is incorporated herein in its entirety.

### FIELD OF THE DISCLOSURE

The field of the disclosure relates generally to cancer treatment.

### BACKGROUND

RAS genes are the most frequently mutated oncogenes in human cancer. Among the RAS isoforms, KRAS is the most frequently mutated (86%), followed by NRAS (11%), which is predominantly mutated in cutaneous melanoma (28%). See: Cox A D, Fesik S W, Kimmelman A C, et al, “Drugging the undruggable RAS: Mission possible?”, Nat Rev Drug Discov 13:828-51, 2014; Hilmi Kodaz, Osman Kostek, Muhammet Bekir Hacioglu, et al., “Frequency of RAS Mutations (KRAS, NRAS, HRAS) in Human Solid Cancer”, EJMO 1:1-7, 2017; and Cancer Genome Atlas N, “Genomic Classification of Cutaneous Melanoma”, Cell 161:1681-96, 2015. Preclinical models of RAS-mutant driven cancers have demonstrated the role of KRAS and NRAS in tumor initiation and maintenance. To date, however, there has been limited clinical success in treating RAS-mutant tumors by targeting its downstream effector pathways, such as the inhibition of PI3K and MEK.

The RAF kinase family, which consist of three subtypes (A-RAF, B-RAF, C-RAF), is a key component of the MAPK signaling pathway downstream of RAS. Mutations in RAF genes, particularly BRAF at codon V600, have been identified in various cancers, including malignant melanoma, colorectal, thyroid, and lung cancers. See Davies H, Bignell G R, Cox C, et al., “Mutations of the BRAF gene in human cancer”, Nature 417:949-54, 200. The BRAF V600 mutations enable BRAF to signal as a monomer, thereby constitutively activating the downstream MAPK signaling pathway.

The discovery of BRAF monomer inhibitors, such as, vemurafenib, dabrafenib, and encorafenib, has led to notable advances in the treatment of patients with BRAF^{V600}-mutant tumors; nevertheless, the durability of treatment response has been limited due to a variety of resistance mechanisms including BRAF amplification, BRAF splice variants and RAS mutations, that largely converge on BRAF dimerization, and resistance to BRAF V600 monomer therapies. See Sullivan R J, Flaherty K T, “Resistance to BRAF-targeted therapy in melanoma” Eur J Cancer 49:1297-304, 2013. Furthermore, these BRAF^{V600} inhibitors have also been shown to paradoxically activate the MAPK signaling pathway in BRAF wild-type and KRAS-mutant cell lines, resulting in the dimerization of BRAF and CRAF, and activation of MEK and ERK signaling in a RAS-dependent manner. See: Heidorn S J, Milagre C, Whittaker S, et al., “Kinase-dead BRAF and oncogenic RAS cooperate to drive tumor progression through CRAF”, Cell 140:209-21, 2010; and Blasco R B, Francoz S, Santamaria D, et al., “c-Raf, but not B-Raf, is essential for development of K-Ras oncogene-driven non-small cell lung carcinoma”, Cancer Cell 19:652-63, 2011. Problematically, 5-20% of patients receiving BRAF^{V600} therapies develop squamous cell carcinomas (SCCs), which is likely driven through the paradoxical activation of the MAPK pathway.

Treatment options for advanced melanoma have improved significantly with the approvals of several immunotherapeutic agents that may be used as monotherapy (e.g., pembrolizumab or nivolumab) or in combination (e.g., ipilimumab plus nivolumab) (Raedler 2015; Ribas et al. 2015; Robert et al. 2019). Based on data from multiple Phase III trials (Seth et al. 2020), these therapies are the recommended initial treatment for BRAF WT melanoma, which includes NRAS-mutant melanoma, in the advanced disease setting. However, there is no clear standard of care following progression on anti-PD-1 agents alone or in combination, and patients are typically treated with further immunotherapy or chemotherapy.

NRAS mutation-positive melanoma has a prevalence of 29% and is a subset of BRAF WT melanoma. There is currently no specific targeted therapy for patients with melanoma harboring NRAS mutations. As such, this patient population has limited treatment options as described above and a high unmet need following progression on or after anti-PD-1 treatment.

A need therefore exists for improved treatments for cancers having KRAS, NRAS and RAF mutations.

### BRIEF DESCRIPTION

In some aspects, the present disclosure is directed to a method for treating a cancer in a human subject, comprising administering an effective amount of belvarafenib to the human subject, wherein the cancer has at least one mutation selected from a BRAF^{V600E} mutation, a BRAF^{G468R} mutation, a BRAF^{V599E} mutation, a KRAS^{G12V} mutation, a KRAS^{G12D} mutation, a KRAS^{G12C} mutation, a KRAS^{G12R} mutation, a KRAS^{G13D} mutation, a KRAS^{Q61H} mutation, a NRAS^{G12D} mutation, a NRAS^{Q61K} mutation, a NRAS^{Q61R} mutation, a NRAS^{Q61L} mutation, a NRAS^{G13D} mutation, and a NRAS^{G12C} mutation.

In some other aspects, the present disclosure is directed to a method for treating cancer in a subject, comprising administering an effective amount of belvarafenib to the subject, wherein the cancer is sarcoma carrying a KRAS^{G12V} mutation, nephroblastoma carrying a BRAF^{V600E} mutation, melanoma carrying a NRAS^{G12D} mutation, melanoma carrying a NRAS^{G12C} mutation, GIST carrying a BRAF^{V600E} mutation, gallbladder cancer carrying a KRAS^{G12D} mutation, CRC carrying a KRAS^{G12C} mutation, CRC carrying a KRAS^{G13D} mutation, CRC carrying a KRAS^{Q61H} mutation, CRC carrying a KRAS^{G12D} mutation, bladder cancer carrying a KRAS^{G12D} mutation, bladder cancer carrying a KRAS^{G12V} mutation, thyroid cancer carrying a BRAF^{V600E} mutation, thyroid cancer carrying a BRAF^{G468R} mutation, thyroid cancer carrying a KRAS^{G12R} mutation, and any of the combinations thereof.

### BRIEF DESCRIPTION OF THE DRAWINGS

FIG. 1 is a CONSORT diagram of the patient disposition in the dose-escalation phase. In the dose-escalation phase, the Full Analysis Set (FAS, efficacy population) included sixty seven of 72 patients. Five patients without any post-dose tumor response assessments due to withdrawal of consent (n=2), adverse event (n=2), or progression of disease or lack of treatment effect (n=1) were excluded from the FAS.

FIG. 2 is a CONSORT diagram of the patient disposition in the dose-expansion phase. In the dose-expansion phase, FAS included fifty-seven of 63 patients. Four patients without any post-dose tumor response assessments due to violation of inclusion/exclusion criteria (n=1) or confirmed PD or lack of efficacy in the judgement of the investigator (n=3) were excluded from the FAS.

FIG. 3 is a plot of the best tumor response percentage changes in the size of target lesions from baseline and the specific genetic mutations in each evaluable patient in the dose-escalation phase.

FIG. 4 is presents plots of progression-free survival of all patients in the dose-escalation phase (FIG. 4A), progression free survival of NRASm melanoma patients in the dose-escalation phase (FIG. 4B), progression-free survival of all patients in the dose-expansion phase (FIG. 4C), and progression free survival of NRASm melanoma patients in the dose-expansion phase (FIG. 4D).

FIG. 5 is a plot of the best tumor response percentage changes in the size of target lesions from baseline and the specific genetic mutations in each evaluable patient in the dose-expansion phase.

FIG. 6 is a plot of treatment duration with the time to partial response in the dose-escalation phase.

FIG. 7 is a plot of treatment duration with the time to partial response in the dose-expansion phase.

FIGS. 8A and 8B are plots of vemurafenib and belvarafenib, respectively, for inhibition of cell viability in BRAF V600 mutant, NRAS mutant, KRAS mutant, and RAS/RAF wild type tumor cell lines.

FIG. 9 depicts the results of a clonogenic assay for the inhibition of colony growth of belvarafenib at varying concentrations in NRAS mutant and BRAF mutant cells.

FIG. 10A is a plot of vemurafenib inhibition of cell viability in cell lines bearing a BRAF^{V600} mutation, bearing a NRAS mutation, bearing a KRAS mutation, or RAS/RAF wild type. FIG. 10B is a plot of belvarafenib inhibition of cell viability in cell lines bearing a BRAF^{V600} mutation, bearing a NRAS mutation, bearing a KRAS mutation, or RAS/RAF wild type.

FIG. 11 is a plot of belvarafenib inhibition of cell viability in BRAF V600E mutant, NRAS mutant, and RAS/RAF wild type melanoma cell lines.

FIG. 12A is a plot of the results of a mutant melanoma syngeneic model study of A375 tumor volume (mm³) over a 29 day treatment period for daily treatment with dabrafenib and for daily treatment with belvarafenib. FIG. 12B is a plot of the results of a mutant melanoma syngeneic model study of HCT-116 tumor volume (mm³) over a 14 day treatment period for daily treatment with dabrafenib and for daily treatment with belvarafenib. FIG. 12C is a plot of the results of a mutant melanoma syngeneic model study of SK-MEL-30 tumor volume (mm³) over a 28 day treatment period for daily treatment with dabrafenib and for daily treatment with belvarafenib.

FIG. 13 is a plot of circulating tumor BRAF^{V600E} MAF DNA (ctDNA) levels versus baseline levels in patients having BRAF^{V600E} mutant cancers during the course of treatment with belvarafenib.

FIG. 14 is a plot of circulating tumor KRAS/NRAS MAF levels DNA (ctDNA) versus baseline levels in patients having KRAS and NRAS mutant cancers during the course of treatment with belvarafenib.

FIG. 15A is a plot of circulating tumor BRAF^{V600E} MAF DNA (ctDNA) in patients having BRAF mutant cancer following treatment with belvarafenib. FIG. 15B is a plot of circulating tumor NRAS^{mut} MAF DNA (ctDNA) in patients having NRAS mutant cancer following treatment with belvarafenib. FIG. 15C is a plot of circulating tumor KRAS^{mut} MAF DNA (ctDNA) in patients having KRAS mutant cancer following treatment with belvarafenib.

FIG. 16A shows a CT scan of a patient having NRAS^{Q61R} melanoma at the start of treatment and FIG. 16B shows a CT scan of the patient after 8 weeks of treatment with belvarafenib at a dose of 450 mg BID with a lesion indicated by the arrow.

FIG. 16C shows a CT scan of a patient having BRAF^{V600E} colon cancer at the start of treatment and FIG. 16D shows a CT scan of the patient after 8 weeks of treatment with belvarafenib at a dose of 450 mg BID with a lesion indicated by the arrow.

FIG. 17A presents the BRAF^{V600E} MAF ctDNA results versus belvarafenib treatment cycle time for patients having BRAF^{V600E} colon cancer, BRAF^{V600E} melanoma, and BRAF^{V600E} nephroblastoma where the belvarafenib therapy achieved stable disease or partial response, or where the disease progressed. FIG. 17B presents the NRAS mutant MAF ctDNA results versus belvarafenib treatment cycle time for patients having NRAS^{mut} melanoma and NRAS^{mut} mucosal melanoma where the belvarafenib therapy achieved stable disease or partial response. FIG. 17C presents the KRAS mutant MAF ctDNA results versus belvarafenib treatment cycle time for patients having KRAS′t colon cancer, KRAS^{mut} pancreatic cancer, KRAS^{mut} endometrial cancer where the belvarafenib therapy achieved stable disease, or where the disease progressed.

FIG. 18 is a depiction of a co-crystal structure showing binding of belvarafenib to BRAF.

FIG. 19A is a depiction of a co-crystal structure showing binding of the RAF inhibitors vemurafenib, dabrafenib, and encorafenib to BRAF. FIG. 19B is a depiction of a co-crystal structure showing binding of the pan-RAF inhibitors belvarafenib and LXH-254 to BRAF.

### DETAILED DESCRIPTION

In accordance with the present disclosure, it has been discovered that the compound belvarafenib is a highly potent and selective type II RAF dimer inhibitor (a pan-RAF inhibitor) that provides for selective inhibition of BRAF and CRAF isoforms. In contrast with BRAF^{V600}-selective monomer inhibitors, it has been discovered that belvarafenib does not activate the MAPK pathway in non-BRAF^{V600} mutant cells, but instead sustains the suppression of MAPK signaling by inhibiting BRAF and CRAF dimers, and results in reduced cell proliferation and increased antitumor activity in both BRAF^{V600}- and RAS-mutant tumors. It has further been discovered that belvarafenib is well-tolerated in human subjects. It has further been discovered that belvarafenib therapy may be done in the absence of the development of squamous cell carcinoma. It has been further discovered that belvarafenib is effective for the treatment of melanoma where, prior to said belvarafenib treatment, the subject experienced disease progression after treatment with immunotherapy, BRAF^{V600E} therapy, or a combination of immunotherapy and BRAF^{V600E} therapy.

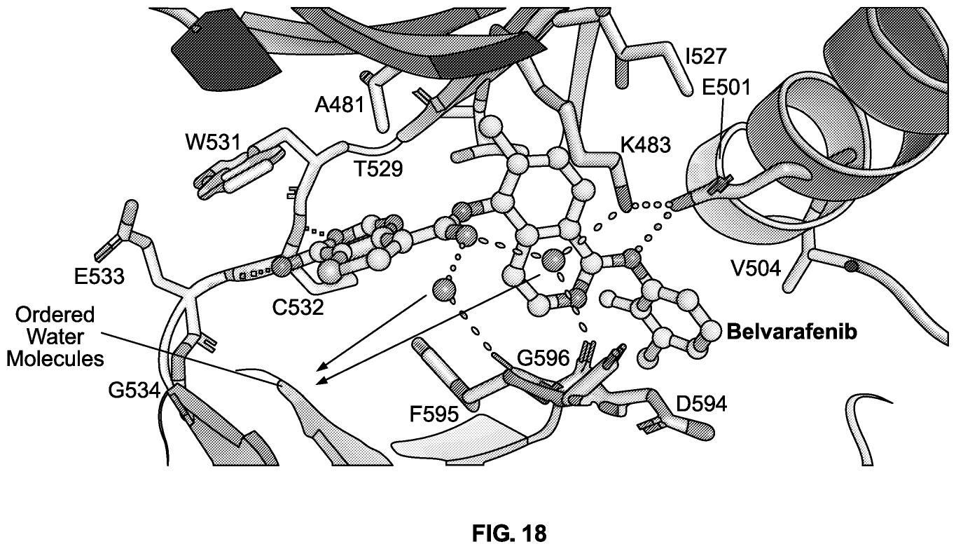

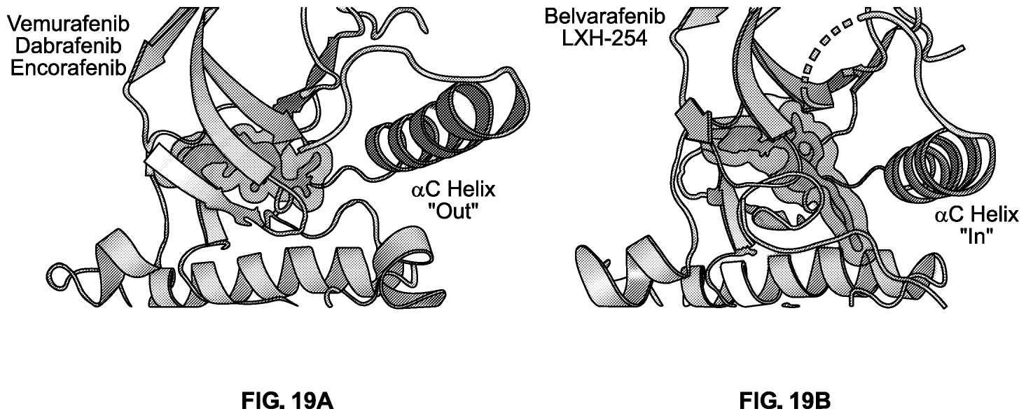

Without being bound to any particular binding theory, FIGS. 18 and 19B depict certain co-crystal structures of the pan-RAF inhibitor belvarafenib bound to BRAF. FIG. 19A is a depiction of a co-crystal structure showing binding of the RAF inhibitors vemurafenib, dabrafenib, and encorafenib to BRAF.

Belvarafenib is disclosed in PCT application WO 2013/100632, has the chemical name 4-amino-N-(1-((3-chloro-2-fluorophenyl)amino)-6-methylisoquinolin yl)thieno[3,2-d]pyrimidine-7-carboxamide (referred to herein as Formula (I)), and has the following chemical structure:

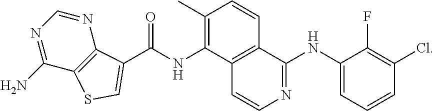 [SMILES: Cc1ccc2c(Nc3cccc(Cl)c3F)nccc2c1NC(=O)c1csc2c(N)ncnc12]

Belvarafenib has been discovered to be effective for treatment of certain cancers in a subject. A subject within the scope of the disclosure is a mammal, including, but not limited to, a human or non-human mammal, such as a bovine, equine, canine, sheep or feline. In some aspects, the subject is a human.

Belvarafenib may suitably be in the form of stereoisomers, geometric isomers and tautomers, and solvates, metabolites, isotopes, pharmaceutically acceptable salts, or prodrugs thereof. In some particular aspects, belvarafenib is a pharmaceutically acceptable salt thereof. As used herein, the term “pharmaceutically acceptable salts” refers to those salts which retain the biological effectiveness and properties of the free bases or free acids, and which are not biologically or otherwise undesirable. Exemplary acid salts of belvarafenib include, but are not limited, to sulfate, citrate, acetate, oxalate, chloride, bromide, iodide, nitrate, phosphate, acid phosphate, lactate, salicylate, acid citrate, tartrate, tartrate, ascorbate, succinate, maleate, fumarate, gluconate, glucuronate, formate, benzoate, glutamate, methanesulfonate “mesylate”, ethanesulfonate, benzenesulfonate, and p-toluenesulfonate. In some aspects, the salt is selected from group consisting of the bis-hydrochloride salt, the bis-hydrogensulfate salt, the bis-p-toluenesulfonate salt, the bis-ethanesulfonate salt, and the bis-methanesulfonate salt. In some aspects, the salt is the bis-hydrochloride salt or the bis-methanesulfonate salt. In one aspect, the salt is the bis-methanesulfonate salt.

Belvarafenib may suitably be either in amorphous or crystalline forms. In some aspects the salt is crystalline. In some such aspects, the salt is the bis-methanesulfonate salt. In some particular aspects, the bis-methanesulfonate salt is characterized by a powder X-ray diffraction (PXRD) pattern having one, two, three, four, five, six, seven, eight, nine or ten peaks, three or more peaks, or five or more peaks selected from those at diffraction angle 2θ±0.2° values of 5.6°, 7.1°, 7.6°, 11.4°, 15.1°, 15.4°, 16.6°, 18.2°, 20.4°, 21.5°, 22.3°, 22.7°, 23.1°, 24.4°, 24.9° and 25.6°, when irradiated with a Cu-Kα light source. In some aspects the salt is the bis-hydrochloride salt. In some particular aspects, the bis-hydrochloride salt is polymorph Form I characterized by a powder X-ray diffraction pattern having three or more peaks selected from those at diffraction angle 2θ values of 5.89°±0.2°, 7.77°±0.2°, 8.31°±0.2°, 11.80°±0.2°, 16.68°±0.2°, 23.22°±0.2°, 23.69°±0.2°, 26.89°±0.2°, 27.51°±0.2°, and 29.53°±0.2°, when irradiated with a Cu-Kα light source. The solid form (crystalline or amorphous) may suitably be determined by PXRD recorded in a D8 ADVANCE made by BRUKER AXS in Germany, operating at 25° C. and at 40.0 KV and 100 mA, using Cu Kα (1.54056 Å) line and rotation.

Belvarafenib may suitably be formulated with one or more pharmaceutically acceptable carriers, adjuvants, and/or excipients and in the form of a capsule, tablet (pill), powder, syrup, dispersion, suspension, emulsion, solution, or the like. Non-limiting examples of suitable liquid carriers include water; saline; aqueous dextrose; glycols; ethanol; oils including those of petroleum, animal, vegetable or synthetic origin; and combinations thereof. Non-limiting examples of suitable pharmaceutical adjuvants/excipients include starch, cellulose (e.g., microcrystalline cellulose), polyvinylpyrrolidone, crospovidone, croscarmellose sodium, talc, D-mannitol, glucose, lactose, talc, gelatin, fumaric acid, fumarate, malt, rice, flour, chalk, silica, magnesium stearate, sodium stearate, sodium stearyl fumarate, glycerol monostearate, sodium chloride, dried skim milk, glycerol, propylene glycol, water, ethanol, and the like, and combinations thereof. Belvarafenib may also be suitably formulated with additional conventional pharmaceutical additives such as preservatives, stabilizing agents, wetting or emulsifying agents, salts for adjusting osmotic pressure, buffers and the like. Such compositions will, in any event, contain an effective amount of belvarafenib so as to prepare the proper dosage form for proper administration to the subject. Belvarafenib may be suitably administered to the subject orally.

In some aspects, belvarafenib may be in the form of film-coated tablets for oral administration. Such suitable tablets may comprise 50 mg, 100 mg, or 150 mg of belvarafenib on a free base equivalent basis. In some such aspects, the tablets comprise belvarafenib and the inactive ingredients D-mannitol, fumaric acid, crospovidone, magnesium stearate (vegetable), sodium stearyl fumarate, and film coating mixture. In some such aspects, tablets comprise belvarafenib and the inactive ingredients microcrystalline cellulose, lactose, croscarmellose sodium, magnesium stearate and film coating mixture. Film coatings are known in the art. In some aspects, the film coating mixture may suitably comprise polyvinyl alcohol, titanium dioxide, macrogol/polyethylene glycol, talc, and iron oxide yellow. In some aspects, the active ingredient comprises, consists essentially of, or consists of belvarafenib, such as for instance belvarafnib·2HCl.

In any of the various aspects of the disclosure, the cancer may be melanoma, nephroblastoma, gastrointestinal stromal tumors (GIST), colorectal cancer (CRC), sarcoma, gallbladder cancer, bladder cancer, and any combinations thereof. In one aspect, the cancer is melanoma. In one aspect, the cancer is nephroblastoma. In one aspect, the cancer is GIST. In one aspect, the cancer is CRC. In one aspect, the cancer is sarcoma. In one aspect, the cancer gallbladder cancer. In one aspect, the cancer is bladder cancer.

In some aspects, the cancer is sarcoma carrying a KRAS^{G12V} mutation, a nephroblastoma carrying a BRAF^{V600E} mutation, melanoma carrying a NRAS^{G12D} mutation, melanoma carrying a NRAS^{Q61K} mutation, melanoma carrying a NRAS^{Q61R} mutation, melanoma carrying a NRAS^{G12C} mutation, melanoma carrying a BRAF^{V600E} mutation, GIST carrying a BRAF^{V600E} mutation, gallbladder cancer carrying a KRAS^{G12D} mutation, CRC carrying a BRAF^{V600E} mutation, CRC carrying a KRAS^{G12C} mutation, CRC carrying a KRAS^{G13D} mutation, CRC carrying a KRAS^{Q61H} mutation, CRC carrying a KRAS^{G12D} mutation, bladder cancer carrying a KRAS^{G12D} mutation, bladder cancer carrying a KRAS^{G12V} mutation, thyroid cancer carrying a BRAF^{V600E} mutation, thyroid cancer carrying a BRAF^{G468R} mutation, thyroid cancer carrying a KRAS^{G12R} mutation, and any combination thereof.

In some aspects, the cancer is sarcoma carrying a KRAS^{G12V} mutation, nephroblastoma carrying a BRAF^{V600E} mutation, melanoma carrying a NRAS^{G12D} mutation, melanoma carrying a NRAS^{G12C} mutation, GIST carrying a BRAF^{V600E} mutation, gallbladder cancer carrying a KRAS^{G12D} mutation, CRC carrying a KRAS^{G12C} mutation, CRC carrying a KRAS^{Q61H} mutation, CRC carrying a KRAS^{G12D} mutation, CRC carrying a KRAS^{G13D} mutation, bladder cancer carrying a KRAS^{G12D} mutation, bladder cancer carrying a KRAS^{G12V} mutation, and any of combination thereof.

In some aspects, the cancer is sarcoma carrying a KRAS^{G12V} mutation, melanoma carrying a NRAS^{G12D} mutation, melanoma carrying a NRAS^{Q61K} mutation, melanoma carrying a NRAS^{Q61R} mutation, melanoma carrying a BRAF^{V600E} mutation, GIST carrying a BRAF^{V600E} mutation, and any combination thereof.

In some aspects, the cancer has at least one mutation selected from a KRAS^{G12V} mutation, a KRAS^{G12D} mutation, a KRAS^{G12C} mutation, a KRAS^{G12R} mutation, a KRAS^{Q61H} mutation, a NRAS^{G12D} mutation, a NRAS^{G13D} mutation, a NRAS^{Q61K} mutation, a NRAS^{Q61L} mutation, a NRAS^{Q61R} mutation, a BRAF^{V600E} mutation, a BRAF^{G468R} mutation, a BRAF^{V599E} mutation, a NRAS^{Q61L} mutation, a NRAS^{G12C} mutation, and a KRAS^{G13D} mutation. In some aspects, the cancer has at least one mutation selected from a KRAS^{G12V} mutation, a KRAS^{G12D} mutation, a KRA^{G12C} mutation, a KRAS^{Q61H} mutation, a NRAS^{G12D} mutation, a NRAS^{Q61K} mutation, a NRAS^{Q61R} mutation, and a NRAS^{G12C} mutation.

In some aspects the cancer has two mutations, such as a BRAF mutation and a NRAS mutation, a BRAF mutation and a KRAS mutation, or a KRAS mutation and a NRAS mutation. In one aspect, the cancer has a BRAF mutation and a NRAS mutation. In one such aspect, the cancer has a BRAF^{V600E} mutation and a NRAS^{Q61L} mutation.

In some aspects of the disclosure, a pharmaceutical composition for treating a cancer, comprising an effective amount of belvarafenib is provided. In some such aspects, the cancer has at least one mutation selected from sarcoma carrying a KRAS^{G12V} mutation, nephroblastoma carrying a BRAF^{V600E} mutation, melanoma carrying a NRAS^{G12D} mutation, melanoma carrying a NRAS^{Q61K} mutation, melanoma carrying a NRAS^{Q61R} mutation, melanoma carrying a NRAS^{G12C} mutation, melanoma carrying a BRAF^{V600E} mutation, GIST carrying a BRAF^{V600E} mutation, gallbladder cancer carrying a KRAS^{G12D} mutation, CRC carrying a BRAF^{V600E} mutation, CRC carrying a KRAS^{G12C} mutation, CRC carrying a KRAS^{G13D} mutation, CRC carrying a KRAS^{Q61H} mutation, CRC carrying a KRAS^{G12D} mutation, bladder cancer carrying a KRAS^{G12D} mutation, bladder cancer carrying a KRAS^{G12V} mutation, thyroid cancer carrying a BRAF^{V600E} mutation, thyroid cancer carrying a BRAF^{G468R} mutation, thyroid cancer carrying a KRAS^{G12R} mutation, and any combination thereof. In some such aspects, the cancer is sarcoma carrying a KRAS^{G12V} mutation, nephroblastoma carrying a BRAF^{V600E} mutation, melanoma carrying a NRAS^{G12D} mutation, melanoma carrying a NRAS^{G12C} mutation, GIST carrying a BRAF^{V600E} mutation, gallbladder cancer carrying a KRAS^{G12D} mutation, CRC carrying a KRAS^{G12C} mutation, CRC carrying a KRAS^{Q61H} mutation, CRC carrying a KRAS^{G12D} mutation, CRC carrying a KRAS^{G13D} mutation, bladder cancer carrying a KRAS^{G12D} mutation, bladder cancer carrying a KRAS^{G12V} mutation, and any of combination thereof. In some such aspects, the cancer is sarcoma carrying a KRAS^{G12V} mutation, melanoma carrying a NRAS^{G12D} mutation, melanoma carrying a NRAS^{Q61K} mutation, melanoma carrying a NRAS^{Q61R} mutation, melanoma carrying a BRAF^{V600E} mutation, GIST carrying a BRAF^{V600E} mutation, and any combination thereof. In some such aspects, the cancer has at least one mutation selected from a KRAS^{G12V} mutation, a KRAS^{G12D} mutation, a KRAS^{G12C} mutation, a KRAS^{GUR} mutation, a KRAS^{G13D} mutation, a KRAS^{Q61H} mutation, a NRAS^{G12D} mutation, a NRAS^{G13D} mutation, a NRAS^{Q61K} mutation, a NRAS^{Q61L} mutation, a NRAS^{Q61R} mutation, a BRAF^{V600E} mutation, a BRAF^{G468R} mutation, a BRAF^{V599E} mutation, a NRAS^{Q61L} mutation, and a NRAS^{G12C} mutation. In some aspects, the cancer has at least one mutation selected from a KRAS^{G12V} mutation, a KRAS^{G12D} mutation, a KRAS^{G12C} mutation, a KRAS^{Q61H} mutation, a NRAS^{G12D} mutation, a NRAS^{Q61K} mutation, a NRA^{Q61R} mutation, and a NRAS^{G12C} mutation. In some such aspects, the cancer has two mutations, such as a BRAF mutation and a NRAS mutation, a BRAF mutation and a KRAS mutation, or a KRAS mutation and a NRAS mutation. In one aspect, the cancer has a BRAF mutation and a NRAS mutation. In one such aspect, the cancer has a BRAF^{V600E} mutation and a NRAS^{Q61L} mutation. In any such composition aspects, the cancer may be melanoma, nephroblastoma, gastrointestinal stromal tumors (GIST), colorectal cancer (CRC), sarcoma, gallbladder cancer, bladder cancer, and any combinations thereof. In one aspect, the cancer is melanoma. In one such aspect, the cancer is nephroblastoma. In one such aspect, the cancer is GIST. In one such aspect, the cancer is CRC. In one such aspect, the cancer is sarcoma. In one such aspect, the cancer gallbladder cancer. In one such aspect, the cancer is bladder cancer.

The belvarafenib dose may range from a dose sufficient to elicit a response to the maximum tolerated dose. For instance, and without being bound to any particular dose, the daily dose may suitably be 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, or 1300 mg and any rage constructed therefrom, such as from 100 mg to 1300 mg, from 200 mg to 1300 mg, from 600 mg to 1300 mg, from 700 mg to 1200 mg, or from 800 mg to 1000 mg. Belvarafenib can be dosed once per day, twice per day, three times per day, or four times per day. In some aspects, belvarafenib is dosed once per day. In some aspects, belvarafenib is dosed twice per day. In one aspect, belvarafenib may be dosed at 450 mg BID. Dosing may be done with our without food. The dosing schedule may suitably be every day of a 28-day schedule, or 21 or more days of a 28-day schedule.

### EXAMPLES

### Example 1

Phase I dose-escalation (NCT02405065) and dose-expansion studies (NCT02405065) were conducted in patients with locally advanced and/or metastatic solid tumors carrying mutations in the BRAF, KRAS, and/or NRAS genes. These studies demonstrated the safety, tolerability, and early clinical efficacy of belvarafenib in multiple types of cancers carrying RAS and/or BRAF mutations

Eligible patients had measurable or evaluable disease per the Response Evaluation Criteria in Solid Tumors version 1.1 (RECIST v1.1). See Eisenhauer E A, Therasse P, Bogaerts J, et al., “New response evaluation criteria in solid tumours: revised RECIST guideline (version 1.1)”, Eur J Cancer 45:228-47, 2009. All patients had progressed on one or more prior lines of therapy or had no available standard therapy at the time of study entry. Additional eligibility criteria included Eastern Cooperative Oncology Group (ECOG) performance status ≤2 and life expectancy ≥12 weeks. Patients with cardiovascular abnormalities as mean QTcF>440 msec were excluded from the dose-expansion phase.

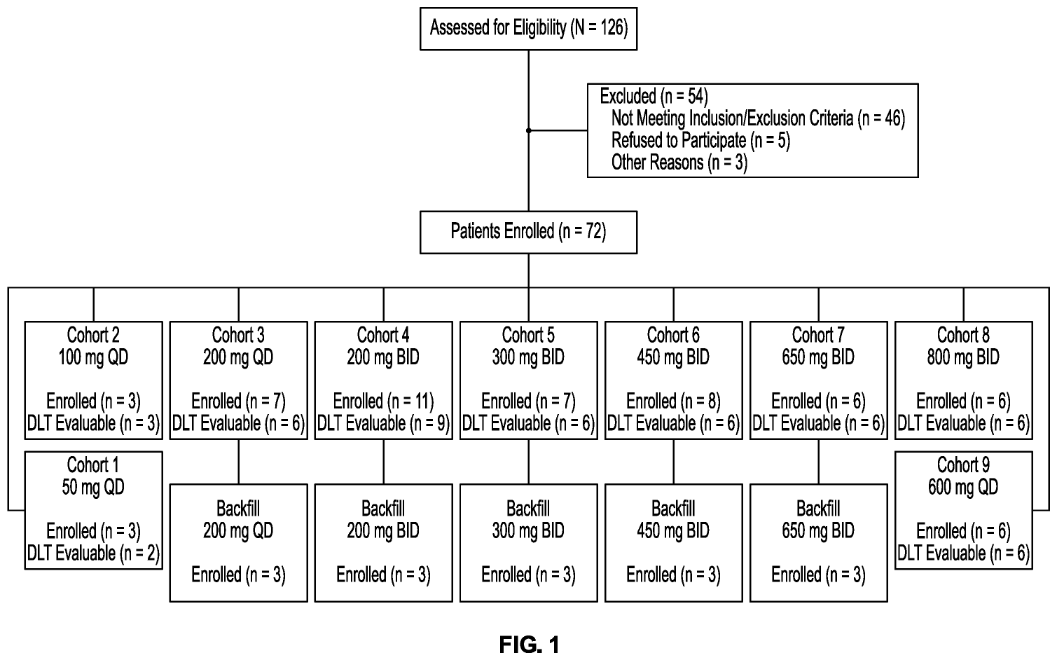

Dose escalation of belvarafenib was carried out using the PK-guided rapid escalation method until the first dose-limiting toxicity (DLT) was observed, followed by the modified Fibonacci scheme of rolling six design (FIG. 1). At dose levels not exceeding the maximum tolerated dose (MTD), additional patients who were able to provide on-treatment tumor biopsies were enrolled in the backfill cohorts to acquire additional pharmacodynamic (PD) data.

Patients received belvarafenib by oral administration at the dose level assigned from 50 mg once daily (QD) up to 800 mg twice daily (BID). The starting dose of belvarafenib was chosen as 50 mg QD, which is the human equivalent dose of the one-tenth the severely toxic dose in 10% of animals (STD₁₀) in rats from pre-clinical studies. See Administration USDoHaHSFaD, “S9 Nonclinical Evaluation for Anticancer Pharmaceuticals”, 2010. Cycle 1 began with a pharmacokinetic (PK) evaluation in which patients received a single dose of belvarafenib on day 1 at their assigned dose level followed by a 7 day washout period. Subsequent treatment cycles were 21 days of continuous dosing.

DLTs were determined during the first cycle. At the end of each dose cohort, the safety and PK data were reviewed for DLT evaluation and the decision whether to continue dose escalation to a subsequent dose level was made. Us used herein, DLT is defined as a toxicity assessed as unrelated to the disease under investigation or disease progression, DLT assessment was performed in Cycle 1 (Dose Escalation Cohorts) according to NCI-CTCAE, version 4.03. An assessment is considered acceptable when the drug compliance during the 21 consecutive days of Cycle 1 is at least 80%.

Non-hematological toxicity is indicated by the following. Grade ≥3 toxicities except for alopecia. Grade ≥3 nausea or vomiting despite antimetic treatment at the highest does. Grade ≥3 diarrhea despite antidiarrheal treatment at the highest dose. Grade ≥3 infection accompanied with grade 4 neutropenia (ANC<500/mm³). QT_{c} prolongation (>500 msec or increase of >60 msec from baseline).

Hematological toxicity is indicated by the following. Grade 4 neutropenia (ANC<500/mm³) persisted for ≥7 days. Grade 4 neutropenia (ANC<500/mm³) accompanied with fever of ≥38.5° C. Grade 4 thrombocytopenia (PLT<25,000/mm³) persisted for >4 days.

Insufficient exposure to treatment is indicated by the following. ≥2 weeks dose delay due to toxicity. Drug compliance of <80% due to toxicity of belvarafenib in 21 consecutive days.

Other toxicities are indicated as follows. A confirmed corneal ulcer. A toxicity which is more severe than baseline level, clinically relevant, refractory to supportive care, and determined as a DLT at SRM.

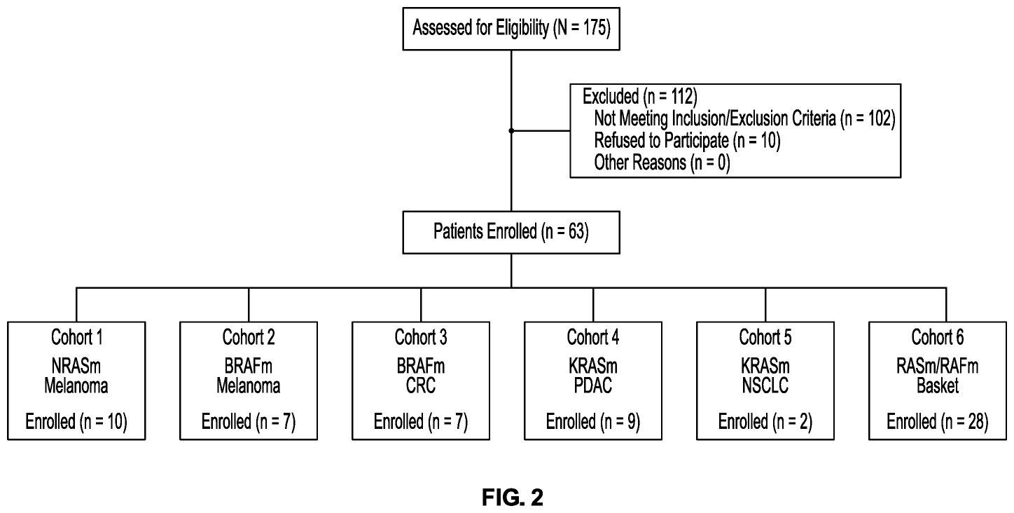

The dose-expansion phase was designed to further evaluate the anti-tumor activity of belvarafenib in patients with specific cancer types and consisted of six cohorts: NRAS-mutant (NRA Sm) melanoma, BRAF-mutant (BRAFm) melanoma, BRAFm colorectal cancer (CRC), KRAS-mutant (KRASm) non-small cell lung cancer (NSCLC), KRASm pancreas ductal adenocarcinoma (PDAC), and a basket cohort of patients with other BRAF or RAS mutation-positive cancers (FIG. 2). Patients received belvarafenib at an oral dose of 450 mg BID in continuous 28-day cycles, which was the recommended dose (RD) determined in the dose-escalation phase. All patients remained in the study until the met a criterion for discontinuation such as disease progression or intolerable toxicity.

The study was conducted in accordance with the provisions of the Declaration of Helsinki, Good Clinical Practice guidelines, and an assurance filed with and approved by the local health authority. The protocols were approved by the institutional review boards at each participating site. Written informed consent was obtained from all participants before the initiation of any study procedures.

In dose-escalation, the DLTs were evaluated by the protocol-specified definitions. Per the protocol, dose escalation was permitted if there was no DLT in three patients or less than one DLT in six patients. If more than two out of six patients experienced DLTs, the dose level was considered not tolerated and the next-lower dose was determined as the MTD. RD determination was done based on the comprehensive evaluation of cumulative data of efficacy, safety, tolerability, and PK/PD from patients in the dose-escalation phase.

Adverse events (AEs) were recorded by the incidence, severity, and relatedness of AEs, and graded per National Cancer Institute-Common Terminology Criteria for Adverse Events (NCI-CTCAE) version 4.03. Safety and tolerability of belvarafenib were evaluated based on AEs, vital signs, physical examinations, electrocardiograms, echocardiogram (ECHO)/multiple-gated acquisition (MUGA) scans, and laboratory tests.

Tumor response assessments were performed radiographically by the investigator using RECIST version 1.1 at baseline, and at the end of every two treatment cycles until discontinuation. Safety evaluation (Safety Set) included all patients who received one or more doses of belvarafenib and efficacy evaluation (Full Analysis Set) included subjects who received at least one dose of belvarafenib and had at least one post-dose tumor response assessment.

Blood samples were collected pre-dose and post-dose at protocol-defined time points for PK assessments of belvarafenib. Full PK analyses were performed to estimate the PK parameters, including AUC₀₋ₗₐₛₜ, AUC_{0-∞}, AUC₀₋₂₄, Cₘₐₓ, Tₘₐₓ, Vₛₛ/F, CL/F, and t_{1/2}. Pharmacodynamic (PD) assessment was performed retrospectively on archival or fresh tumor tissues and blood samples collected from patients. MAPK pathway inhibition by belvarafenib was determined by measuring the expression of MAPK pathway genes as well as changes in pMEK and pERK levels by immunohistochemistry in tumor tissues.

A total of 135 patients were enrolled in the phase I study including 72 patients in the dose-escalation phase and 63 patients in the dose-expansion phase. Patient demographics and baseline characteristics are summarized in Table 1. In Table 1: “ECOG” refers to Eastern Cooperative Oncology Group; “CRC” refers to Colorectal Cancer; “PDAC” refers to Pancreatic Ductal Adenocarcinoma; “NSCLC” refers to Non-small-cell lung carcinoma; “GIS” refers to Gastrointestinal stromal tumor; “Others” includes gallbladder (n=2), malignant neoplasm (n=1), nephroblastoma (n=1), thymic (n=1), ampulla of vater (n=2), cholangiocarcinoma (n=2), breast (n=1), and endometrial (n=1). In “Mutation type, n (%)”, one patient in each phase had mutation in both BRAF and NRAS genes.

TABLE 1

| | | |
|---|---|---|
| Baseline | Dose-escalation | Dose-expansion |
| Characteristic | (N = 72) | (N = 63) |

Age (years)

| | | |
|---|---|---|
| Median (Min, Max) | 58 (31, 78) | 57 (24, 75) |
| Sex, n (%) | | |
| Male | 45 (62.5) | 34 (54.0) |
| Female | 27 (37.5) | 29 (46.0) |

ECOG at screening, n (%)

| | | |
|---|---|---|
| 0 | 16 (22.2) | 26 (41.3) |
| 1 | 52 (72.2) | 37 (58.7) |
| 2 | 4 (5.6) | 0 |

Number of prior therapies, n (%)

| | | |
|---|---|---|
| ≤2 | 39 (54.2) | 37 (58.7) |
| 3-7 | 33 (45.8) | 26 (41.3) |

Primary site of tumor, n (%)

| | | |
|---|---|---|
| CRC | 42 (58.3) | 20 (31.8) |
| Melanoma | 25 (34.7) | 17 (27.0) |
| PDAC | 0 | 10 (15.9) |
| NSCLC | 2 (2.8) | 3 (4.8) |
| Bladder | 1 (1-4) | 2 (3.2) |
| GIST | 1 (1-4) | 0 |
| Sarcoma | 1 (1-4) | 0 |
| Others | 0 | 11 (17.5) |

Mutation type, n (%)

| | | |
|---|---|---|
| KRAS | 30 (41.7) | 30 (47.6) |
| BRAF | 29 (40.3) | 20 (31.7) |
| NRAS | 14 (19.4) | 14 (22.2) |

Median follow up time (weeks)

| | | |
|---|---|---|
| Median (Min, Max) | 16.1 (1.3, 68.5) | 12.4 (0.1, 104.1) |

Among the 72 patients in the dose-escalation phase, 57 patients were enrolled in the dose-escalation cohorts and 15 patients in the backfill cohorts (FIG. 1). The median number of prior therapies was 3 (range, 0-7). In terms of mutations, 29 patients had tumors carrying mutations in BRAF, 30 patients had mutations in KRAS, and 14 patients had mutations in NRAS, including one patient had both BRAF and NRAS mutations and was counted for both groups. The most common types of cancer were colorectal cancer (42 patients) and melanoma (25 patients).

In the dose-expansion phase, 63 patients were enrolled in six pre-specified cohorts based on the locally tested mutational status and tumor type (FIG. 2): NRASm melanoma (10 patients), BRAFm melanoma (7 patients), BRAFm CRC (7 patients), KRASm PDAC (9 patients), KRASm NSCLC (2 patients), and a basket cohort of any other RAS- or RAF-mutant solid tumors (28 patients).

In the dose-escalation phase, fifty patients were considered evaluable for dose determination per protocol. Out of the 57 patients in the dose escalation cohorts, 7 patients who had less than 80% compliance without toxicity including those who withdrew from the study during cycle 1 were excluded from the DLT assessment. Four of the 50 patients experienced DLTs including grade 3 rash in three patients (at 200 mg BID, 650 mg BID, and 800 mg BID), and grade 2 dermatitis acneiform leading to less than 80% of drug compliance in one patient (at 800 mg BID). All DLTs were reversible after belvarafenib interruption and/or concomitant medication. At 800 mg BID, two out of 6 patients experienced DLTs; therefore, 650 mg BID was considered as the MTD for belvarafenib. Following an overall assessment of the tolerability, safety, efficacy, and PK data, the RD for single-agent belvarafenib in further studies was determined as 450 mg twice per day (BID).

The overall safety summary of dose-escalation and dose-expansion (n=135) is shown in Table 2. The most frequently reported treatment-emergent adverse events (TEAEs) across all doses were dermatitis acneiform (37.0%), rash (23.7%), and pruritus (22.2%). At the RD of 450 mg BID, dose reduction occurred in 15 (20.3%) of 74 patients, of which 12 (16.2%) were due to adverse drug reactions (ADRs), and 21 (28.4%) of 74 patients required dose interruptions, of which 17 (23.0%) were due to ADRs. Three patients (4.1%) permanently discontinued treatment because of grade 3 cholangitis, grade 4 hyperkalemia, or grade 4 dermatitis acneiform. In Table 2: “TEAE” refers to treatment-emergent adverse event; “ADR” refers to adverse drug related. The majority of grade 3/4 ADRs were dermatological toxicities that were reversible and manageable with supportive care. No cases of squamous cell carcinoma (SCC) were reported. Serious TEAEs occurred in 30 patients (22.2%), of which 12 (8.9%) were related to belvarafenib.

TABLE 2

| | Total | | 450 mg BID | |
|---|---|---|---|---|
| | All Grades | Grade ≥3 | All Grades | Grade ≥3 |
| Any TEAEs, n (%) | 127 (94.1) | 45 (33.3) | 72 (97.3) | 30 (40.5) |
| TEAE leading to drug | 29 (21.5) | 13 (9.6) | 21 (28.4) | 10 (13.5) |
| Interrupted | | | | |
| TEAE leading to drug | 25 (18.5) | 7 (5.2) | 15 (20.3) | 5 (6.8) |
| reduced | | | | |
| TEAE leading to study | 12 (8.9) | 8 (5.9) | 3 (4.1) | 3 (4.1) |
| drug discontinuation | | | | |
| Any ADRs, n (%) | 113 (83.7) | 26 (19.3) | 62 (83.8) | 16 (21.6) |
| ADR leading to drug | 25 (18.5) | 9 (6.7) | 17 (23.0) | 7 (9.5) |
| Interrupted | | | | |
| ADR leading to drug | 21 (15.6) | 5 (3.7) | 12 (16.2) | 3 (4.1) |
| reduced | | | | |
| ADR leading to study | 9 (6.7) | 8 (5.9) | 3 (4.1) | 3 (4.1) |
| drug discontinuation | | | | |
| Dermatitis acneiform | 50 (37.0) | 4 (3.0) | 34 (46.0) | 4 (5.4) |
| Rash | 32 (23.7) | 4 (3.0) | 16 (21.6) | 2 (2.7) |
| Pruritus | 30 (22.2) | 0 | 18 (24.3) | 0 |
| Decreased appetite | 25 (18.5) | 1 (0.7) | 15 (20.3) | 1 (1.4) |
| Pyrexia | 24 (17.8) | 0 | 15 (20.3) | 0 |
| Nausea | 21 (15.6) | 1 (0.7) | 12 (16.2) | 1 (1.4) |
| Fatigue | 20 (14.8) | 1 (0.7) | 12 (16.2) | 1 (1.4) |
| Constipation | 18 (13.3) | 0 | 15 (20.3) | 0 |
| Vomiting | 18 (13.3) | 2 (1.5) | 9 (12.2) | 2 (2.7) |
| Dyspepsia | 15 (11.1) | 2 (1.5) | 10 (13.5) | 0 |
| Abdominal pain | 14 (10.4) | 0 | 10 (13.5) | 0 |

The PK parameters of belvarafenib were estimated in 48 patients in the dose-escalation phase and 35 patients in the dose-expansion phase. The results are presented in Tables 3 and 4 below. In the tables: “AUCₗₐₛₜ” refers to the area under the plasma concentration-time curve from zero time until the last measurable concentration; “AUC_{0-∞}” refers to area under the plasma concentration-time curve from zero time to infinity; “AUC₂₄” refers to the area under the curve from T₀ to T₂₄; “Cₘₐₓ” refers to the maximal concentration; “Tₘₐₓ” refers to the time to reach Cₘₐₓ; “T_{1/2β}” refers to the terminal elimination half-life; “QD” refers to once a day; and “BID” refers to twice a day. AUC was calculated based on the concentrations measured from 0 (predose) through 48 hours in cohort 1 and from 0 through 168 hours in the other cohorts. Mean and coefficient of variation are presented except for Tₘₐₓ where median and range are presented. One patient in the 200 QD dosing regimen who took concomitant rifampin was removed from the analysis because the AUC and Cₘₐₓ of the patient were much lower than those of the others in the same cohort, which, without being bound to any particular theory, could have resulted from the drug-metabolizing enzyme induction by rifampin through which belvarafenib is biotransformed.

Belvarafenib plasma target exposure was achieved from 200 mg BID and the mean plasma concentration showed linearity in a dose-proportional manner from 50 mg QD to 650 mg BID. Single-dose median Tₘₐₓ was 3.0-4.5 h (QD) and 15.5-24.0 h (BID), and the median t_{1/2} at steady state was 65.1-106.1 h (QD) and 32.3-66.4 h (BID). At 450 mg BID in the dose-expansion phase, the median exposure of 35 patients was similar to the median observed for the same dose level in dose-escalation and consistent with the findings that efficacious exposure was reached at 450 mg BID. To confirm on-target and pathway inhibition by belvarafenib, patient tissues were analyzed for MAPK pathway effectors including pMEK and pERK; as a result, decreases in pMEK and pERK were observed in patients treated with belvarafenib (data not shown).

TABLE 3
First administration (Day 1)

| Dose per | 50 | 100 | 200 | 200 mg | 400 mg |
|---|---|---|---|---|---|
| day (mg) | (n = 3) | (n = 3) | (n = 7) | (n = 6) | (n = 11) |
| Dose | QD | QD | QD | QD | BID |
| Regimen | | | | | |
| AUCₗₐₛₜ | 12957.9 | 44998.5 | 25080.5 | 28805.0 | 62327.8 |
| (μg · hr/L) | (70.7) | (54.6) | (77.9) | (64.1) | (65.1) |
| AUC_{0→∞} | 26992.9 | 51512.2 | 32820.2 | 37767.7 | 78336.7 |
| (μg · hr/L) | (68.8) | (58.7) | (89.5) | (76.3) | (65.4) |
| Cₘₐₓ(μg/L) | 552.7 | 806.7 | 549.4 | 601.8 | 899.8 |
| | (52.5) | (22.8) | (71.3) | (66.7) | (72.7) |
| Cₘₐₓ/Dose | 11.1 | 8.1 | 2.7 | 3.0 | 2.4 |
| (μg/L/min) | (52.5) | (22.8) | (71.3) | (66.7) | (68.5) |
| Tₘₐₓ (h) | 3.0 | 3.0 | 4.1 | 4.4 | 6.9 |
| | (2.9, 5.0) | (3.0, 12.0) | (3.0, 12.0) | (3.0, 12.0) | (3.0, |
| | | | | | 23.9) |
| V_{z}/F (L) | 186.1 | 214.9 | 550.3 | 529.1 | 758.1 |
| | (58.1) | (93.0) | (38.7) | (42.5) | (75.8) |
| CL/F (L/h) | 2.5 | 2.5 | 15.4 | 7.3 | 7.8 |
| | (60.8) | (64.4) | (140.2) | (51.4) | (77.3) |
| T_{1/2β} (h) | 51.6 | 55.3 | 50.5 | 57.7 | 70.7 |
| | (6.8) | (42.9) | (61.0) | (46.0) | (26.2) |
| Dose per | 600 | 900 | 1300 | 1600 mg | 600 mg |
| day (mg) | (n = 7) | (n = 8) | (n = 6) | (n = 6) | (n = 6) |
| Dose | BID | BID | BID | BID | QD |
| Regimen | | | | | |
| AUCₗₐₛₜ | 120886.1 | 1239111.8 | 155869.5 | 134174.6 | 71118.5 |
| (μg · hr/L) | (66.5) | (45.8) | (52.2) | (84.6) | (53.7) |
| AUC_{0→∞} | 169243.6 | 177316.6 | 225005.8 | 335830.8 | 84711.4 |
| (μg · hr/L) | (62.7) | (55.1) | (66.0) | (136.9) | (56.2) |
| Cₘₐₓ | 1213.1 | 1592.3 | 1790.0 | 1438.8 | 1317.8 |
| (μg/L) | (62.7) | (39.2) | (22.1) | (65.0) | (44.8) |
| Cₘₐₓ/Dose | 1.9 | 2.0 | 1.4 | 0.9 | 2.2 |
| (μg/L/min) | (67.2) | (26.4) | (22.1) | (65.0) | (44.8) |
| Tₘₐₓ (h) | 24.0 | 17.0 | 15.5 | 20.4 | 4.5 |
| | (13.0, | (3.1, 23.7) | (3.0, 24.0) | (3.0, | (2.0, 6.9) |
| | 47.9) | | | 169.5) | |
| V_{z}/F (L) | 883.7 | 534.2 | 708.4 | 1698.4 | 797.8 |
| | (94.4) | (33.1) | (32.5) | (65.7) | (72.8) |
| CL/F (L/h) | 7.7 | 5.7 | 9.0 | 14.5 | 9.9 |
| | (99.9) | (60.3) | (79.5) | (85.0) | (64.1) |
| T_{1/2β} (h) | 86.6 | 84.3 | 77.4 | 215.8 | 58.9 |
| | (31.6) | (67.3) | (53.9) | (171.7) | (30.3) |

TABLE 4
Multiple administrations
(Day 17 for cohort 1 and Day 22 for all other cohorts)

| Dose per | 50 | 100 | 200 | 200 mg | 400 mg |
|---|---|---|---|---|---|
| day (mg) | (n = 2) | (n = 3) | (n = 6) | (n = 5) | (n = 8) |
| Dose | QD | QD | QD | QD | BID |
| Regimen | | | | | |
| AUCₗₐₛₜ | 18908.7 | 41851.7 | 41617.2 | 48150.2 | 67531.4 |
| (μg · hr/L) | (5.5) | (60.8) | (76.0) | (63.4) | (51.5) |
| AUC_{0→∞} | 88753.7 | 349410.1 | 203427.4 | 239193.5 | 206835.1 |
| (μg · hr/L) | (10.7) | (100.8) | (93.5) | (78.9) | (64.2) |
| AUC₂₄ | 18931.1 | 41952.4 | 41679.9 | 48215.6 | 68914.7 |
| (μg · hr/L) | (5.4) | (60.8) | (76.1) | (63.5) | (52.5) |
| Cₘₐₓ (μg/L) | 1115.0 | 1986.7 | 2213.0 | 2524.0 | 3213.8 |
| | (3.2) | (55.3) | (72.7) | (62.8) | (49.4) |
| Cₘₐₓ/Dose | 22.3 | 19.9 | 11.1 | 12.6 | 8.0 |
| (μg/L/min) | (3.2) | (55.3) | (72.7) | (62.8) | (49.4) |
| Tₘₐₓ (h) | 3.0 | 5.0 | 3.1 | 3.2 | 13.1 |
| | (1.9, 4.0) | (0.0, 6.0) | (2.0, 6.1) | (3.0, 6.1) | (0.0, 24.0) |
| V_{z}/F (L) | 57.6 | 62.7 | 177.8 | 116.1 | 157.3 |
| | (6.3) | (44.9) | (91.2) | (56.2) | (60.9) |
| CL/F (L/h) | 0.6 | 0.9 | 2.4 | 1.3 | 3.3 |
| | (10.7) | (126.2) | (120.2) | (58.4) | (82.6) |
| T_{1/2β} (h) | 70.7 | 106.1 | 65.1 | 69.8 | 41.2 |
| | (4.5) | (67.1) | (36.2) | (32.9) | (41.5) |
| Dose per | 600 | 900 | 1300 | 1600 mg | 600 mg |
| day (mg) | (n = 6) | (n = 7) | (n = 6) | (n = 4) | (n = 6) |
| Dose | BID | BID | BID | BID | QD |
| Regimen | | | | | |
| AUCₗₐₛₜ | 115975.3 | 126133.9 | 157669.3 | 49224.8 | 57594.4 |
| (μg · hr/L) | (45.1) | (39.9) | (34.5) | (64.2) | (44.7) |
| AUC_{0→∞} | 507018.0 | 322397.4 | 625324.6 | 138758.7 | 434408.2 |
| (μg · hr/L) | (107.2) | (62.8) | (52.7) | (54.0) | (129.0) |
| AUC₂₄ | 120115.1 | 126837.1 | 158708.9 | 49586.6 | 58007.9 |
| (μg · hr/L) | (47.9) | (39.1) | (33.1) | (64.1) | (44.5) |
| Cₘₐₓ (μg/L) | 5695.0 | 6094.3 | 7325.0b | 2362.5 | 3133.3 |
| | (44.8) | (35.5) | (31.1) | (64.9) | (40.3) |
| Cₘₐₓ/Dose | 9.5 | 6.8 | 5.6 | 1.5 | 5.2 |
| (μg/L/min) | (44.8) | (35.5) | (31.1) | (64.9) | (40.3) |
| Tₘₐₓ (h) | 3.4 | 2.9 | 2.0 | 5.5 | 4.1 |
| | (0.0, 23.8) | (09, 16.0) | (0.0, 23.9) | (2.9, 15.0) | (1.1, 12.2) |
| V_{z}/F (L) | 122.3 | 148.4 | 194.8 | 871.3 | 240.6 |
| | (43.1) | (32.5) | (45.1) | (45.8) | (47.4) |
| CL/F (L/h) | 2.2 (63.3) | 3.8 (50.7) | 2.7 (54.7) | 14.8 (61.2) | 3.4 (81.8) |
| T_{1/2β} (h) | 52.3 | 32.3 | 66.4 | 42.7 | 97.7 |
| | (64.4) | (40.8) | (85.4) | (26.9) | (95.8) |

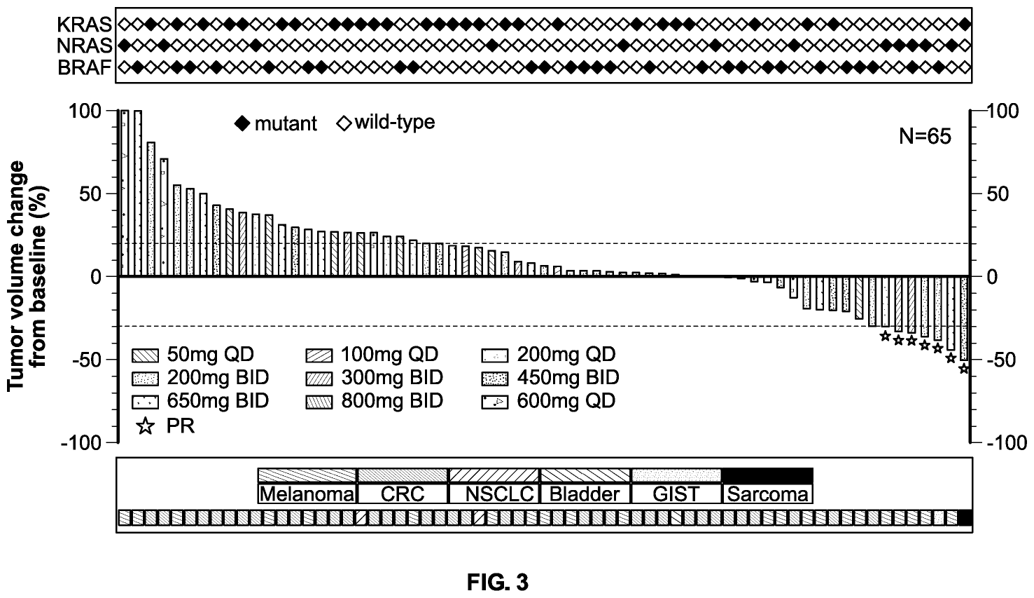

In the dose-escalation phase, tumor response to belvarafenib was assessed in 67 patients who had at least one post-dose tumor response assessment. Tumor response was observed from the dose level of 200 mg QD. Seven patients (10.4%) achieved the best overall response of partial response (PR) and 27 patients (40.3%) achieved stable disease (SD) (Table 3, FIG. 3). The partial response results from FIG. 3 are summarized in Table 5 below.

TABLE 5

| Mutant Type | Cancer Type | Dose | Best Change |
|---|---|---|---|
| KRAS^{G12V} | Sarcoma | 450 mg BID | −50.5% |
| NRAS^{G12D} | Melanoma | 450 mg BID | −44.4% |
| BRAF^{V600E} | GIST | 450 mg BID | −38.1% |
| NRAS^{Q61K} | Melanoma | 450 mg BID | −36.2% |
| NRAS^{Q61} AND BRAF^{V600E} | Melanoma | 450 mg BID | −33.8% |
| BRAF^{V600E} | Melanoma | 450 mg BID | −33.2% |
| NRAS^{Q61R} | Melanoma | 450 mg BID | −30.6% |

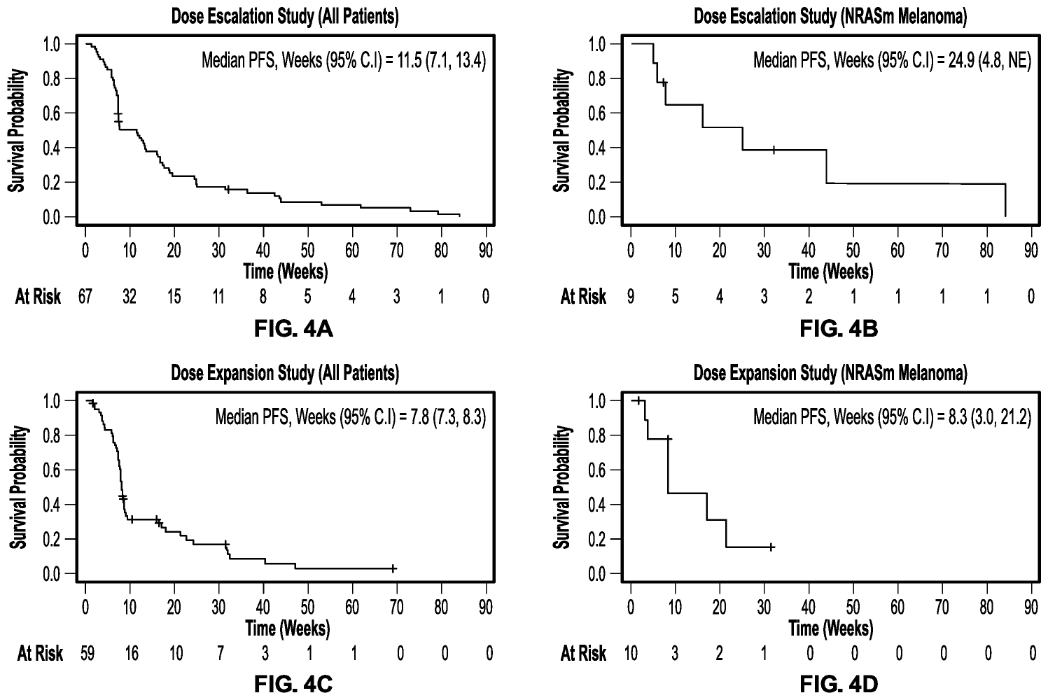

Among the seven responders, four patients were NRAS-mutant melanoma (44% out of the 9 enrolled NRASm melanoma patients) with a median progression-free survival (mPFS) of 25 weeks (95% CI, 4.8 to not estimable). See Table 6 below and FIG. 4). In Table 6: “pts” refers to patients; “mel.” refers to melanoma; “unconf.” refers to unconfirmed; “conf.” refers to confirmed; “BORR” refers to best overall response rate; “PFS” refers to progression-free survival; “DOR” refers to duration of response; “NE” refers to not estimable. BORR (%)=(Number of subjects with best overall response as CR or PR/Total number of subjects)*100. ORR (%)=(Number of subjects with confirmed best overall response as CR or PR/Total number of subjects)*100. DCR (%)=(Number of subjects with best overall response as CR, PR or SD/Total number of subjects)*100. In the DOR row of Table 6: ^{a} indicates that one patient discontinued due to AE (G2 fatigue, G2 depression); ^{b} indicates that two patients are ongoing; and ^{c} indicates that one patient is ongoing.

TABLE 6
Tumor response in efficacy-evaluable patients from
the dose-escalation and dose-expansion phases.

| | Dose-Escalation Study | | Dose-Expansions Study | | | |
|---|---|---|---|---|---|---|
| | NRASm | All pts | NRASm | BRAFm | BRAFm | All pts |
| | mel. (N = 9) | (N = 67) | mel. (N = 10) | mel. (N = 6) | CRC (N = 6) | (N = 57) |
| BORR | 4 (44.4) | 7 (10.5) | 2 (20.0) | 2 (33.3) | 2 (33.3) | 7 (11.9) |
| (unconf.), n | | | | | | |
| (%) | | | | | | |
| ORR (conf.), | 1 (11.1) | 3 (4.5) | 2 (20.0) | 1 (16.7) | 1 (16.7) | 4 (6.8) |
| n(%) | | | | | | |
| DCR, n(%) | 6 (66.7) | 34 (50.6) | 6 (60.0) | 5 (83.3) | 2 (33.3) | 21 (35.6) |
| PFS | | | | | | |
| Median | 24.9 | 11.5 | 8.3 | 22.5 | 7.5 | 7.8 |
| (weeks) | | | | | | |
| 95% C.I. for | (4.8, NE) | (7.1, 13.4) | (3.0, 21.2) | (8.26, NE) | (3.99, 24.06) | (7.3, 8.3) |
| median | | | | | | |
| DOR, n | 4^{a} | 7 | 2^{b} | 2^{c} | 2 | 4 |
| Median | 17.8 | 24.0 | NE | 22.5 | 13.5 | 15.7 |
| (weeks) | | | | | | |
| 95% C.I. for | (3.3, 37.3) | (3.3, 37.3) | (NE, NE) | (NE, NE) | (10.8, 16.0) | (10.8, 16.0) |
| median | | | | | | |

Three of 4 responders with NRASm melanoma progressed on prior immunotherapy and responded to belvarafenib. See Table 7 below. In Table 7: “PR” refers to partial response; “SD” refers to stable disease; “PD” refers to progressive disease; and “BOR” refers to best overall response.

TABLE 7
NRA-mutant melanoma patients with prior immunotherapy treatments

| Phase | NRAS Mutation | Belvarafenib BOR | Prior Treatment |
|---|---|---|---|
| Dose-escalation | Q61R | PR | Ipilimumab |
| Dose-escalation | Q61K | PR | Nivolumab |
| Dose-escalation | G12D | PR | Ipilimumab |
| Dose-expansion | Q61R | PR | Pembrolizumab |
| Dose-expansion | Q61K | PR | Nivolumab |
| Dose-expansion | Q61L | SD | Pembrolizumab |
| Dose-escalation | G12C | SD | Nivolumab |
| Dose-expansion | Q61R | SD | Pembrolizumab |
| Dose-expansion | Q61R | PD | REGN2810 |
| Dose-escalation | Q61R | PD | Pembrolizumab |
| Dose-expansion | Q61H | PD | Nivolumab |
| Dose-expansion | G12D | PD | Pembrolizumab |

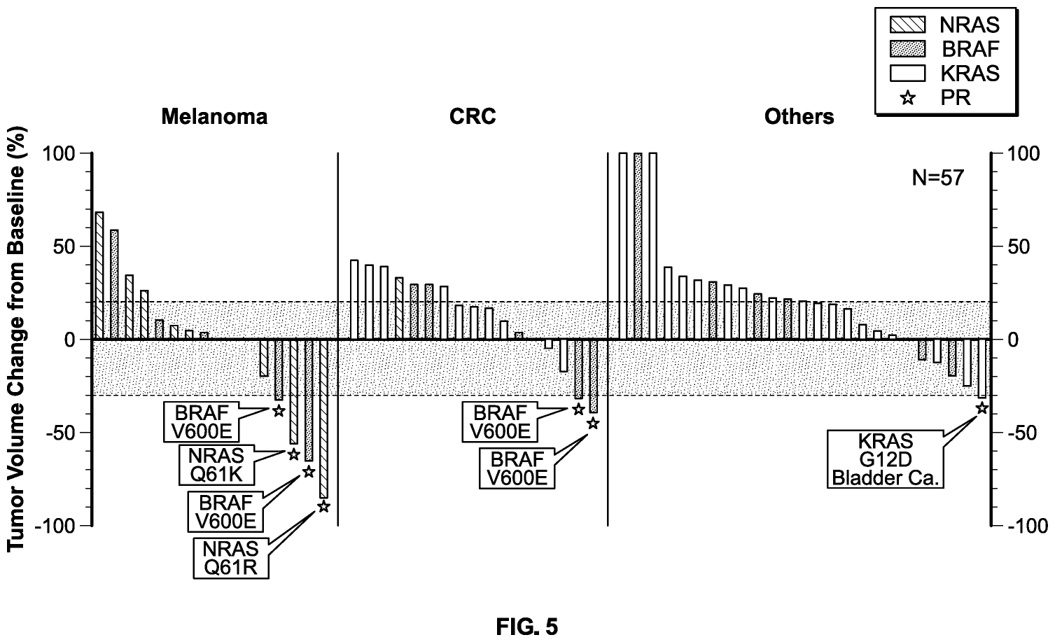

In the dose-expansion phase, among the 10 patients with NRASm melanoma, two patients (20%) achieved PR and four patients (40%) had SD. The disease control rate (DCR: PR+SD) was 60% (See Table 5 above and FIG. 5). The partial response results from FIG. 5 are summarized in Table 8 below.

TABLE 8

| Mutant Type | Cancer Type | Dose | Best Change |
|---|---|---|---|
| NRAS^{Q61R} | Melanoma | 450 mg BID | −84.8% |
| BRAF^{V600E} | Melanoma | 450 mg BID | −70% |
| NRAS^{Q61K} | Melanoma | 450 mg BID | −60% |
| BRAF^{V600E} | CRC | 450 mg BID | −48% |
| BRAF^{V600E} | Melanoma | 450 mg BID | −39% |
| BRAF^{V600E} | CRC | 450 mg BID | −39% |
| KRAS^{G12D} | Bladder | 450 mg BID | −38% |

Two patients with PR were also noted for prior progression on immunotherapy and responded to belvarafenib. In the BRAFm melanoma cohort, two (33%) of 6 patients achieved PR and the DCR was 83%. In BRAFm CRC cohort, two (33%) of 6 patients achieved PR. Disease control (PR or SD) with belvarafenib were observed in 6 patients with BRAF^{V600E}-mutant melanoma or CRC (from dose-escalation and dose-expansion) who progressed in prior BRAF^{V600E} inhibitors. See Table 9 where “BRAFi” refers to BRAF inhibitor; “uPR” refers to unfolded protein response; and “cPR” refers to confirmed partial response. In Table 9, the phase for each patient was the dose-escalation phase and the setting was palliative.

TABLE 9
BRAF^{V600E}-mutant melanoma and CRC patients with prior
BRAF^{V600E} inhibitor treatments

| | Primary | Prior | BOR of | Belvarafenib |
|---|---|---|---|---|
| Mutation | Cancer | BRAFi | prior BRAFi | BOR |
| BRAF ^{V600E} | sigmoid colon | Encorafenib | PD | SD |
| BRAF ^{V600E} | sigmoid colon | Encorafenib | Unknown | SD |
| BRAF ^{V600E} | melanoma | Dabrafenib | Unknown | PD |
| BRAF ^{V600E}, | melanoma | Vemurafenib | PD | uPR |
| NRAS ^{Q61L} | | | | |
| BRAF ^{V600E} | melanoma | Vemurafenib | Unknown | SD |
| BRAF ^{V600E} | melanoma | Vemurafenib | PR | PD |
| BRAF ^{V600E} | melanoma | Dabrafenib | PD | cPR |
| BRAF ^{V600E} | melanoma | Vemurafenib | SD | PD |
| BRAF ^{V600E} | melanoma | Vemurafenib | SD | SD |

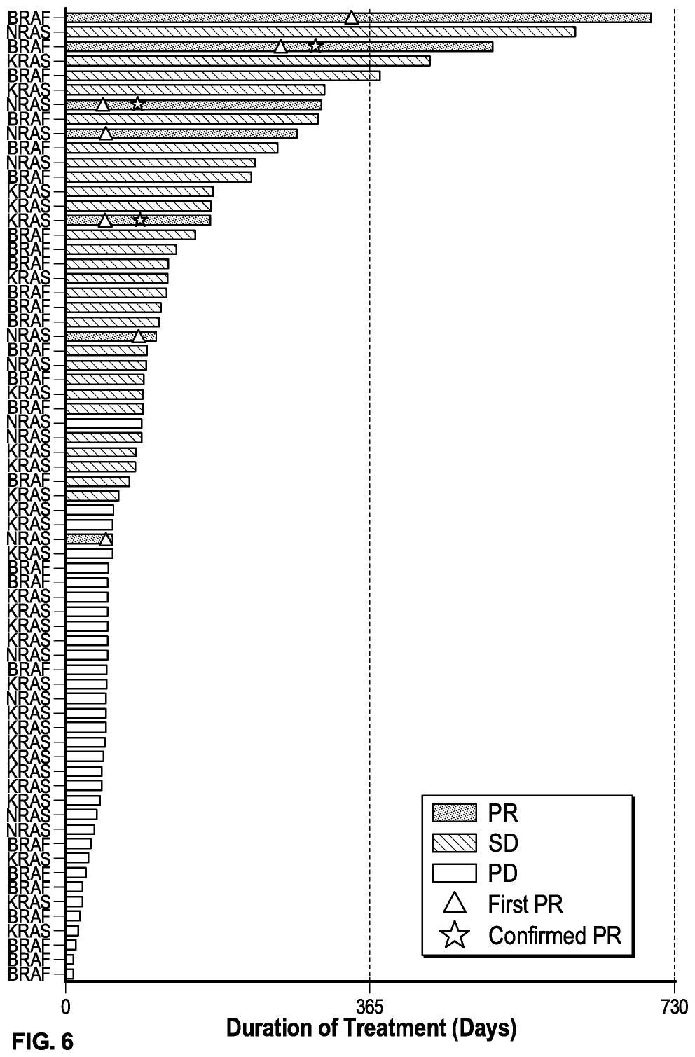

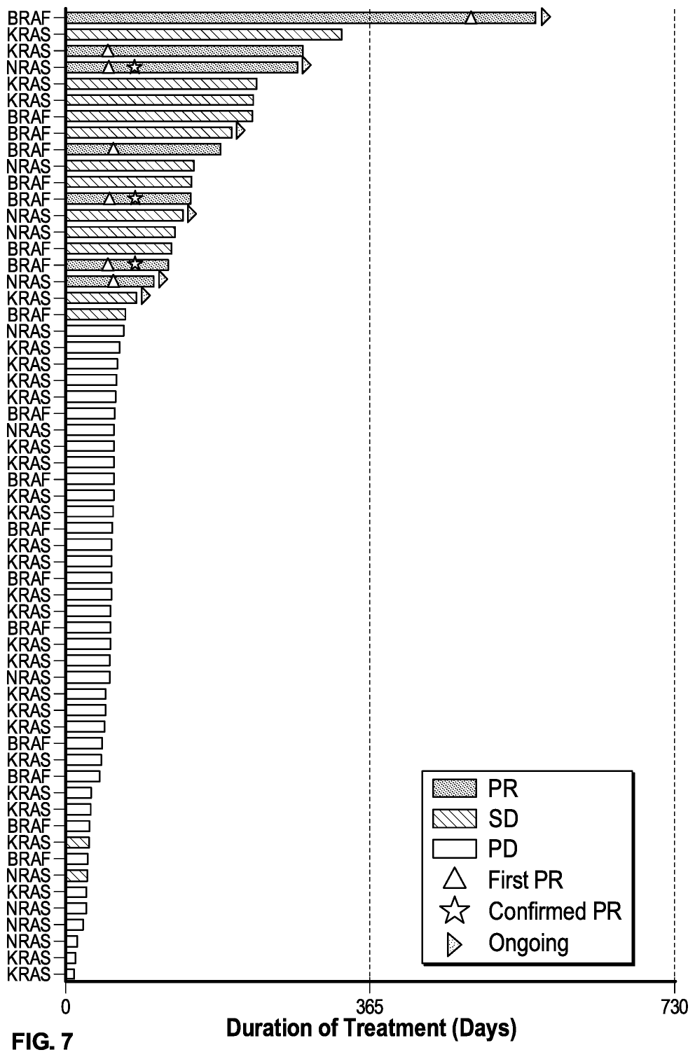

Responses were also noted in patients with BRAFm GIST, KRASm sarcoma, and KRASm bladder cancer (n=1 each) with tumor response durations of 36 weeks, 18 weeks, and 33 weeks, respectively. Additionally, six patients (BRAF^{V600E} melanoma [n=3], NRAS^{G12C} melanoma [n=1], BRAF^{V600E} GIST [n=1], KRAS^{G12C} CRC [n=1]) were maintained on treatment for more than a year. FIG. 6 and FIG. 7 depict the treatment duration with the time to PR in the dose-escalation and dose-expansion phases.

The 10 longest duration treatments of FIG. 6 are summarized in Table 10 below.

TABLE 10

| Mutation Type | Cancer Type | Dose | Duration (days) |
|---|---|---|---|
| BRAF^{V600E} | Melanoma | 800 mg BID | 702 |
| NRAS^{G12C} | Melanoma | 800 mg BID | 611 |
| BRAF^{V600E} | GIST | 200 mg BID | 512 |
| KRAS^{G12C} | CRC | 100 mg BID | 437 |
| BRAF^{V600E} | Melanoma | 450 mg BID | 377 |
| KRAS^{G12V} | Bladder | 300 mg BID | 311 |
| NRAS^{Q61K} | Melanoma | 200 mg BID | 307 |
| BRAF^{V600E} | CRC | 200 mg BID | 303 |
| NRAS^{Q61K} | Melanoma | 300 mg BID | 278 |
| BRAF^{V600E} | CRC | 200 mg BID | 255 |

The 7 longest duration treatments of FIG. 7 are summarized in Table 11 below.

TABLE 11

| Mutation Type | Cancer Type | Dose | Duration (days) |
|---|---|---|---|
| BRAF^{V600E} | Melanoma | 450 mg BID | 563 |
| KRAS^{G12D} | Gallbladder | 450 mg BID | 331 |
| KRAS^{G12D} | Bladder | 450 mg BID | 284 |
| NRAS^{Q61R} | Melanoma | 450 mg BID | 278 |
| KRAS^{Q61H} | CRC | 450 mg BID | 229 |
| KRAS^{G12D} | CRC | 450 mg BID | 225 |
| BRAF^{V600E} | Nephroblastoma | 450 mg BID | 224 |

The results indicate that belvarafenib is generally well-tolerated at RD of 450 mg BID and that the ADRs were primarily grade 1/2, manageable, and reversible with treatment interruption and/or supportive care, as clinically indicated. The most frequently reported TEAEs were dermatological toxicities including dermatitis acneiform, rash, and pruritus. The safety profile of belvarafenib is expected to be comparable to those or other MAPK pathway-targeting inhibitors no cases of SCC were observed with belvarafenib treatment. The development of SCC is observed in clinically approved BRAF inhibitors.

The phase I study of belvarafenib demonstrated clinical activity in patients with NRASm and BRAF^{V600E}-mutant tumors. In particular, the efficacy of belvarafenib observed in NRASm melanoma patients (best overall response rate [BORR] of 44% and PFS of 24.9 weeks in the dose-escalation and BORR of 20% in the dose-expansion, Table 5) provides clinical evidence that NRAS-driven MAPK activation can be effectively inhibited by RAF dimer inhibition. This result also suggests that Type II RAF dimer inhibitors, which effectively block BRAF and CRAF dimers, have clinical activity profiles that are distinct from BRAF^{V600} inhibitors, namely vemurafenib, dabrafenib and encorafenib, that induce paradoxical activation in the context of RAS mutations. Recent clinical data in NRAS-mutant melanoma patients treated with binimetinib reported an overall response rate of 15%, mPFS of 2.8 months and no significant difference in OS compared with dacarbazine control (NEMO study). See Dummer R, Schadendorf D, Ascierto P A, et al., “Binimetinib versus dacarbazine in patients with advanced NRAS-mutant melanoma (NEMO): a multicentre, open-label, randomised, phase 3 trial”, The Lancet Oncology 18:435-445, 2017. The modest response in this subpopulation of melanoma suggests that a stronger suppression of MAPK signaling is required for overall survival benefit and that targeting the MAPK pathway at multiple nodes may provide a more durable efficacy. See Ryan M B, Corcoran R B, “Therapeutic strategies to target RAS-mutant cancers”, Nat Rev Clin Oncol 15:709-720, 2018. Further, a notable response to belvarafenib was observed even in melanoma patients who were previously treated with immunotherapy or who had been treated with prior BRAF^{V600E} therapies and progressed (Tables 6 and 7). Of the 19 NRAS-mutant melanoma patients, 12 had received prior immunotherapy regimens and five responded to belvarafenib after progressing on immunotherapy. Thus, belvarafenib may be a valuable subsequent strategy for melanoma patients who have failed standard immunotherapy regimens.

In addition to the response observed in NRAS-mutant melanoma, responses were observed in three patients with BRAF^{V600E}-mutant melanoma, thus supporting the activity of belvarafenib in MAPK-altered melanoma tumors. Some of these patients had received prior BRAF therapies including vemurafenib and dabrafenib and progressed. In one case, it was observed that a patient carried both BRAF^{V600E} and NRAS mutations; upon closer inspection of this case, it was discovered that the NRAS mutation was acquired after 41 months of treatment with BRAF-targeted therapy. This patient initially achieved a complete response to BRAF-targeted therapy but later developed resistance as evidenced by the subsequent acquisition of NRAS mutation. This patient was treated with belvarafenib for over 9 months and achieved a partial response. BRAF inhibition alone is known to lead to the enrichment of NRASm subclones in tumors and NRAS mutation co-occurs in 14% of patients treated with BRAF^{V600} inhibitors. See Trunzer K, Pavlick A C, Schuchter L, et al., “Pharmacodynamic effects and mechanisms of resistance to vemurafenib in patients with metastatic melanoma”, J Clin Oncol 31:1767-74, 2013. Given the mechanisms of resistance to BRAF therapies largely converge on RAF dimerization in line with the supporting preclinical data of belvarafenib on BRAF therapy-resistant cell lines (see Namgoong G, S. H. Kim T H S, Bae I H, et al: A selective and potent pan-RAF inhibitor, HM95573 exhibits high therapeutic potential as a next-generation RAF inhibitor by direct inhibition of RAF kinase activity in BRAF or RAS mutant cancers. European Journal of Cancer 69:S127, 2016), under one theory and without being bound to any particular theory, the activity of belvarafenib in the aforementioned patients may have be driven through the inhibition of RAF dimers.

The responses observed in heavily pre-treated BRAF-mutant CRC patients (2 of 6 patients; BORR 33%) support the potent inhibitory function of belvarafenib in non-melanoma BRAF-mutant tumors. These results are also in contrast with the historical data of BRAF inhibitor monotherapy in BRAF^{V600E}-mutant CRC tumors that showed 5% to 9% response rate. See: Corcoran R B, Atreya C E, Falchook G S, et al., “Combined BRAF and MEK Inhibition With Dabrafenib and Trametinib in BRAF V600-Mutant Colorectal Cancer”, Journal of Clinical Oncology 33:4023-4031, 2015; Kopetz S, Desai J, Chan E, et al., “Phase II Pilot Study of Vemurafenib in Patients With Metastatic BRAF-Mutated Colorectal Cancer”, Journal of Clinical Oncology 33:4032-4038, 2015; and Falchook G S, Long G V, Kurzrock R, et al., “Dabrafenib in patients with melanoma, untreated brain metastases, and other solid tumours: a phase 1 dose-escalation trial”, Lancet 379:1893-901, 2012. These differences also likely highlight the difference in the mechanism of action between belvarafenib and clinically approved BRAF inhibitors. Improved clinical results (ORR 26% in 111 patients) of the triple combination of encorafenib, binimetinib, and cetuximab in BRAF^{V600}-mutant CRC were recently reported. See Kopetz S, Grothey A, Yaeger R, et al., “Encorafenib, Binimetinib, and Cetuximab in BRAF V600E-Mutated Colorectal Cancer”, N Engl J Med, 2019. However, 58% of the patients receiving the triple combination experienced grade 3 or higher AEs. In contrast, belvarafenib as a single-agent has shown a modest efficacy in the same patient population with a favorable safety profile, which makes it an appropriate candidate for combination with MEK and EGFR inhibitors.

It was observed that belvarafenib provided limited disease control in patients with KRAS-mutant tumors.

The present experimental data indicates that continuous, twice-daily oral treatment with belvarafenib provides for promising clinical treatment activities in NRAS- and BRAF^{V600E}-mutant melanoma, and BRAF^{V600E}-mutant CRC tumors.

### Example 2

Belvarafenib was evaluated to measure the inhibition capability of RAF monomers and dimers. The results are reported in Tables 12 and 13. In Table 12: “A375” refers to the A375 a human melanoma cell line bearing a BRAF^{V600E} mutation; “IPC298” refers to the IPC298 cutaneous melanoma cell line bearing an NRAS^{Q61L} mutation; “A549” refers to the A549 lung adenocarcinoma cell line baring a KRAS mutation; “CSFR1” refers to the CSFR1 gene; “DDR1” refers to discoidin domain receptors DDR1; and “DDR2” refers to discoidin domain receptors DDR2. In Table 13: “% P/T−MEK” refers to the ratio of P-MEK and total MEK; “Conc 1” refers to an inhibitor first (lowest) concentration; “Conc 2” refers to an inhibitor second (intermediate) concentration; and “Conc 3” refers to an inhibitor third (highest) concentration. In Table 13, LXH254 refers to a drug having CAS No.: 1800398-38-2 and the following structure:

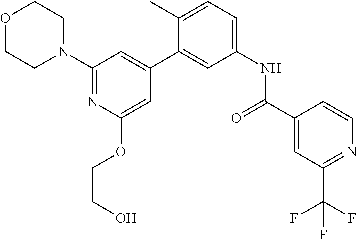 [SMILES: Cc1ccc(NC(=O)c2ccnc(C(F)(F)F)c2)cc1-c1cc(OCCO)nc(N2CCOCC2)c1]

TABLE 12

| Ki CRAF/BRAF/BRAF^{V600E} | 2 nM/41 nM/2 nM |
|---|---|
| A375 IC₅₀ | 0.19 μM |
| IPC298 IC₅₀ | 0.13 μM |
| A549 IC₅₀ | 0.57 μM |
| Kinase selectivity (#inh >80% at 1 μM) | 3/187 (CCSFR1, DDR1, DDR2) |

TABLE 13

| | RAF monomer | RAF dimer |
|---|---|---|
| | inhibitor | inhibitor |
| | % P/T-MEK | % P/T-MEK |
| Vemurafenib Conc1 | 130 | — |
| Vemurafenib Conc2 | 150 | — |
| Vemurafenib Conc3 | 125 | — |
| Dabrafenib Conc1 | 125 | — |
| Dabrafenib Conc2 | 150 | — |
| Dabrafenib Conc3 | 75 | — |
| Belvarafenib Conc1 | — | 75 |
| Belvarafenib Conc2 | — | 60 |
| Belvarafenib Conc3 | — | 20 |
| LXH254 Conc1 | — | 80 |
| LXH254 Conc2 | — | 70 |
| LXH254 Conc3 | — | 10 |

### Example 3

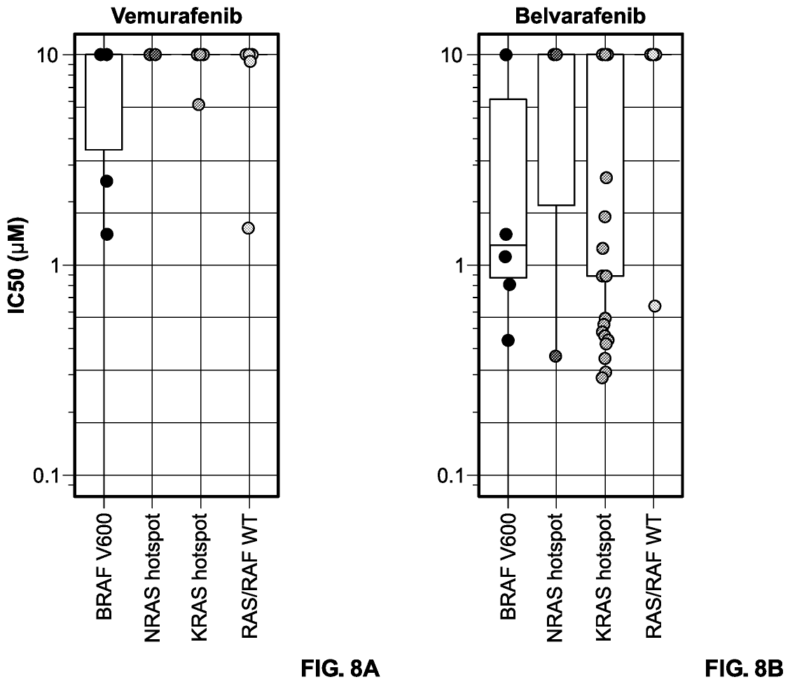

Belvarafenib was evaluated versus vemurafenib for pan-RAF dimer inhibition capability in BRAF and NRAS mutant tumor lines bearing the following mutations: BRAF^{V600}; KRAS hotspot; NRAS hotspot; and RAS/RAF wildtype. Cell screening was done across a panel of 142 cell lines (lung, ovary, colon, breast, brain, gastric, and uterine), including BRAF^{V600} mutant, NRAS mutant, KRAS mutant, and RAS/RAF wild type cells treated with vemurafenib or belvarafenib in 3-day cell viability studies. IC₅₀ values (μM) were determined using a four-parameter fit using nonlinear regression analysis. The results for vemurafenib are shown in FIG. 8A and the results for belvarafenib are shown in FIG. 8B. The results indicate that belvarafenib is a pan-RAF dimer inhibitor that inhibits BRAF^{V600} mutant and NRAS mutant tumor cell lines. Belvarafenib is therefore active in BRAF and NRAS mutant melanoma.

### Example 4

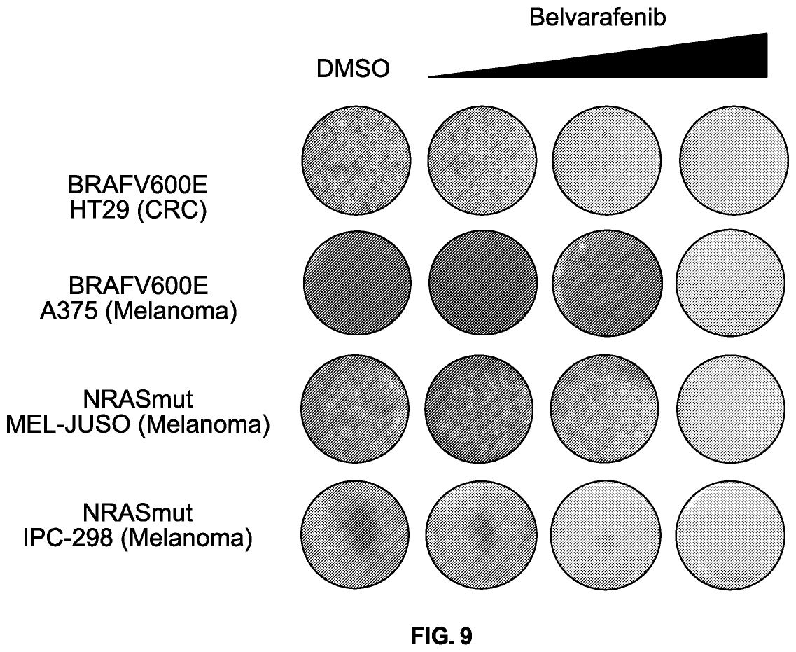

Belvarafenib was evaluated for inhibition capability against NRAS and BRAF in a clonogenic assay. The cells were treated with belvarafenib at four concentrations over a range of concentrations, were cultured for 8 days, and then were stained with crystal violet. The results are shown in FIG. 9 where: “HT29” refers to human colorectal adenocarcinoma cell line HT-29 bearing a BRAF^{V600E} mutation; “A375” refers to a human melanoma cell line bearing a BRAF^{V600E} mutation; “MEL-JUSO” refers to human melanoma cell line MelJuSo bearing a NRAS^{Q61L} mutation; and “IPC-298” refers to a human melanoma cell line bearing an NRAS^{Q61L} mutation. The stained cells at the highest concentration in FIG. 9 correlate with FIG. 8. The results show that belvarafenib inhibits colony cell growth in BRAF^{V600E} and NRAS mutant cell lines in vitro.

### Example 5

Vemurafenib and belvarafenib were evaluated over a concentration range against cell lines bearing a BRAF^{V600} mutation, bearing a NRAS mutation, bearing a KRAS mutation, and bearing a RAS/RAF wild type mutation. Cell screening was done across a panel of 27 skin cell lines including BRAF^{V600} mutant, NRAS mutant, KRAS mutant, and RAS/RAF wild type cell lines treated with vemurafenib or belvarafenib in 3-day cell viability studies. IC₅₀ values (μM) were determined using a four-parameter fit using nonlinear regression analysis. Cell viability data for BRAF^{V600E} mutant, NRAS mutant, and wild type melanoma cell lines was evaluated after 3 days of treatment with belvarafenib.

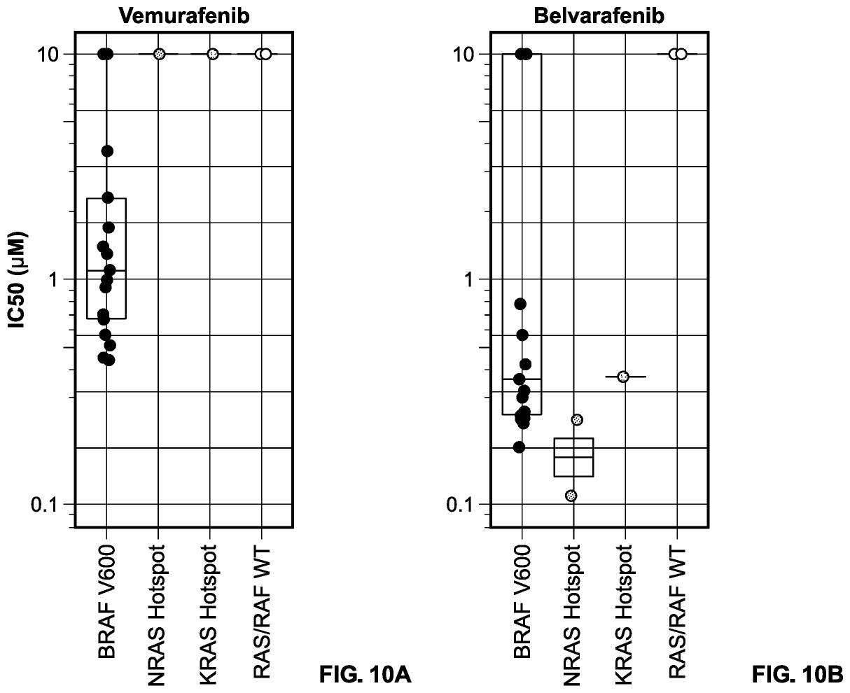

A first set of results in IC₅₀ (μM) are shown in FIG. 10A for vemurafenib and FIG. 10B for belvarafenib.

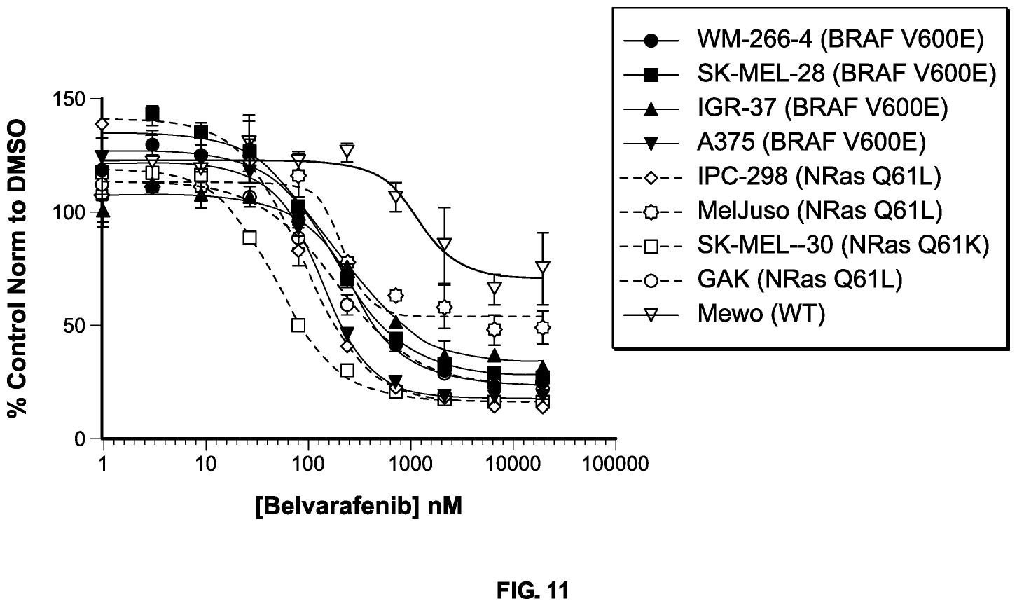

A second set of results for belvarafenib is shown in FIG. 11. Belvarafenib was evaluated over a concentration range of 1 nM to 40,000 nM against cell the following cell lines: WM-266-4, which is a human melanoma cell line bearing a BRAF^{V600E} mutation; SK-MEL-28, which is a human melanoma cell line bearing a BRAF^{V600E} mutation; IGR-37, which is a human melanoma cell line bearing a BRAF^{V600E} mutation; A375, which is a human melanoma cell line bearing a BRAF^{V600E} mutation; IPC-298, which is a human melanoma cell line bearing an NRAS^{Q61L} mutation; MelJuSo, which is a human melanoma cell line bearing a NRAS^{Q61L} mutation; SK-MEL-30 which is a human melanoma cell line bearing a NRAS^{Q61K} mutation; GAK, which is a human melanoma cell line bearing a NRAS^{Q61L} mutation; and Mewo, which is a human melanoma cell wild type cell line. The results are reported in % control normal to DMSO versus concentration.

FIGS. 10A, 10B, and 11 show results that belvarafenib exhibits single agent activity in BRAF^{V600E} mutant and NRAS mutant melanoma tumor cell lines in vitro. The results further show that vemurafenib can inhibit BRAF^{V600E} mutant cell lines but is unable to inhibit NRAS mutant, KRAS mutant, or RAS/RAF wild type cell lines.

### Example 6

In a mutant melanoma syngeneic model study, dabrafenib and belvarafenib were evaluated over time periods for control of A375, HCT-116 and SK-MEL-30 cell lines. HCT-116 is a human colon cancer cell line bearing a KRAS^{G13D} mutation.

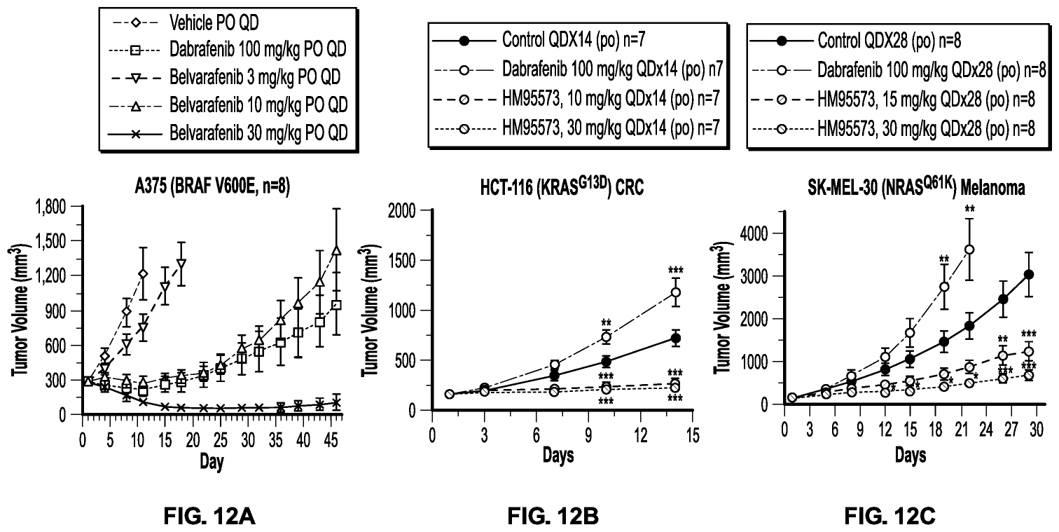

FIG. 12A presents results for A375 tumor volume (mm³) over a 29 day treatment period with the following: oral administration of a control once per day for 29 days, where n=8; oral administration of dabrafenib at 100 mg/kg once per day for 29 days, where n=8; oral administration of belvarafenib (HM95573) at 3 mg/kg once per day for 29 days, where n=8; oral administration of belvarafenib (HM95573) at 10 mg/kg once per day for 29 days, where n=8; and oral administration of belvarafenib (HM95573) at 30 mg/kg once per day for 29 days, where n=8.

FIG. 12B presents results for HCT-116 tumor volume (mm³) over a 14 day treatment period with the following: oral administration of a control once per day for 14 days, where n=7; oral administration of dabrafenib at 100 mg/kg once per day for 14 days, where n=7; oral administration of belvarafenib (HM95573) at 10 mg/kg once per day for 14 days, where n=7; and oral administration of belvarafenib (HM95573) at 30 mg/kg once per day for 14 days, where n=7.

FIG. 12C presents results for SK-MEL-30 tumor volume (mm³) over a 28 day treatment period with the following: oral administration of a control once per day for 28 days, where n=8; oral administration of dabrafenib at 100 mg/kg once per day for 28 days, where n=8; oral administration of belvarafenib (HM95573) at 15 mg/kg once per day for 28 days, where n=8; and oral administration of belvarafenib (HM95573) at 30 mg/kg once per day for 28 days, where n=8.

The results indicate that belvarafenib shows potent in vivo anti-tumor activity against BRAF, KRAS and NRAS mutants. Belvarafenib is more effective at decreasing tumor volume than either the vehicle or dabrafenib in A375 (BRAF^{V600E} mutant) or SK-MEL-30 (NRAS^{Q61K} mutant) xenograft mouse models (n=8/group, mean tumor volume±SEM).

### Example 7

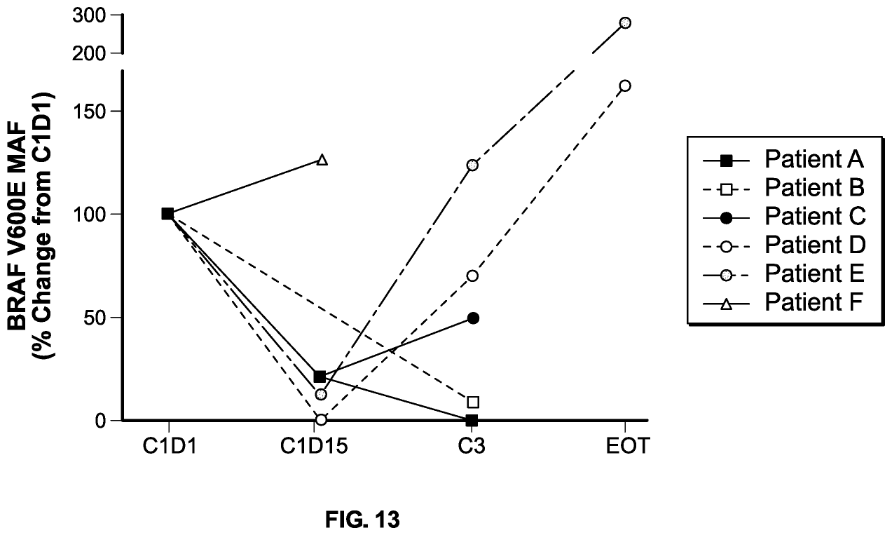

In a clinical evaluation for circulating tumor DNA (ctDNA), belvarafenib was evaluated on patients having BRAF^{V600E} mutant cancers. The results are presented in Table 14 below and in FIG. 13. FIG. 13 presents results in % change in BRAF^{V600E} mutant allele frequency (MAF) as compared to BRAF^{V600E} MAF at C1D1 (cycle 1, day 1). ctDNA was measured by a FoundationACT® blood-based circulating tumor DNA (ctDNA) assay available from Foundation Medicine.

TABLE 14

| | | | | Last |
|---|---|---|---|---|
| Patient | Mutation | Cancer | BOR | Cycle |
| A | BRAF^{V600E} | CRC | PR (−39%) | C3 |
| B | BRAF^{V600E} | Melanoma | PR (−65%) | C3 |
| C | BRAF^{V600E} | Nephroblastoma | SD (−10%) | C3 |
| D | BRAF^{V600E} | Melanoma | SD (11%) | EOT |
| E | BRAF^{V600E} | Melanoma | SD (4%) | EOT |
| F | BRAF^{V600E} | CRC | PD | EOT |

The results show that BRAF^{V600E} allele decreases in all patients that had a clinical response. The results further show that allele frequency increases again after C1D15 (cycle 1, day 15) in patients that has stable disease (SD).

### Example 8

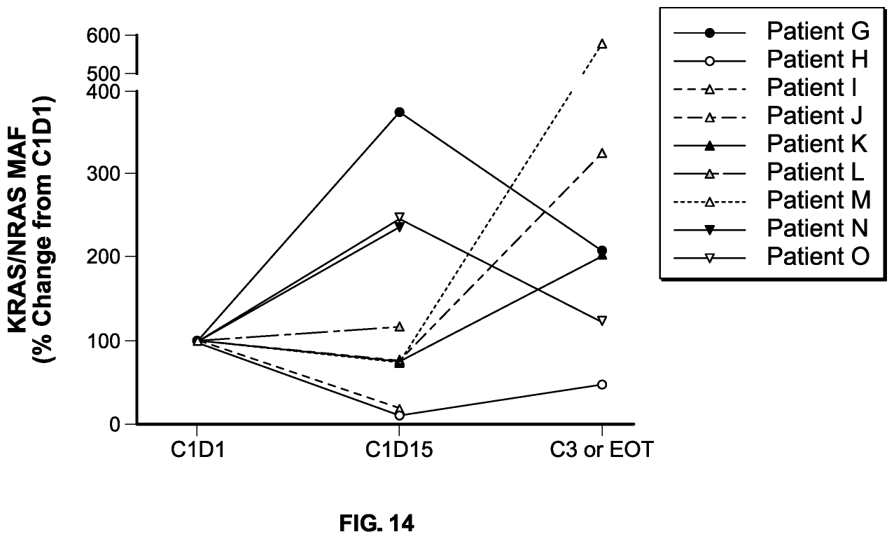

In a clinical evaluation for circulating tumor DNA (ctDNA), belvarafenib was evaluated on patients having KRAS and NRAS mutant cancers. The results are presented in Table 15 below and in FIG. 14. FIG. 14 presents results in % change in KRAS/NRAS MAF as compared to KRAS/NRAS MAF at C1D1 (cycle 1, day 1).

TABLE 15

| | | | | Last |
|---|---|---|---|---|
| Patient | Mutation | Cancer | BOR | Cycle |
| G | KRAS^{Q61L} | CRC | SD (−14%) | C3 |
| H | NRAS^{Q61R} | Melanoma | SD (0%) | C3 |
| I | KRAS^{G12V} | Pancreatic | PD | EOT |
| J | KRAS^{G12V} | Pancreatic | PD | EOT |
| K | KRAS^{G12V} | Pancreatic | PD | EOT |
| L | KRAS^{G12D} | Pancreatic | PD | EOT |
| M | KRAS^{Q61H} | CRC | PD | EOT |
| N | KRAS^{G12D} | Pancreatic | PD | EOT |
| O | KRAS^{G12V} | Pancreatic | PD | EOT |

The results show that the RAS allele is stable or increases with treatment.

### Example 9

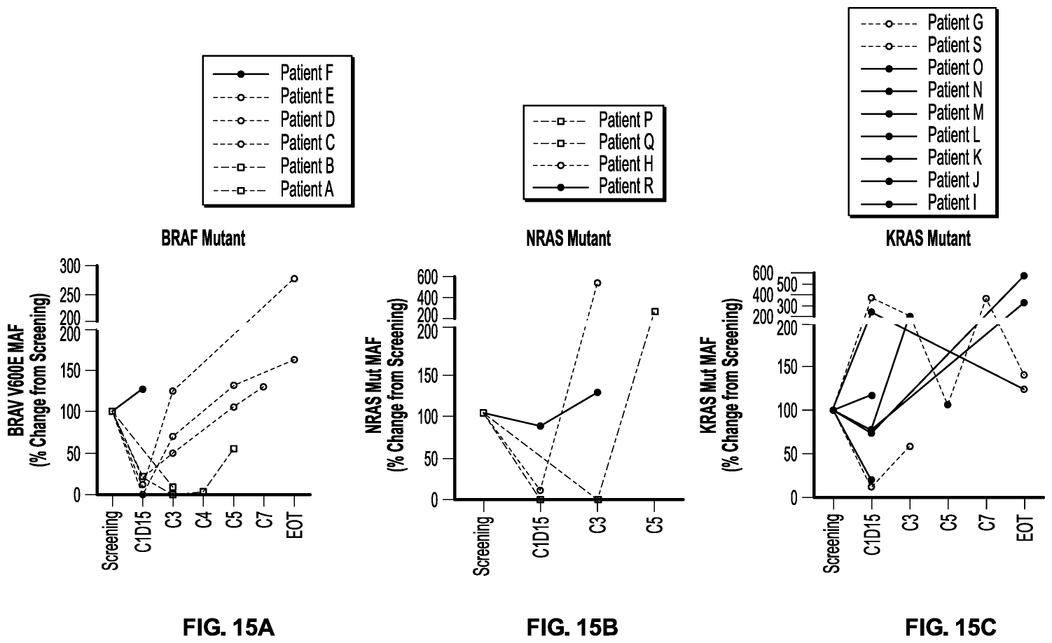

In a clinical evaluation for circulating tumor DNA (ctDNA), belvarafenib was evaluated on patients having BRAF, NRAS and KRAS mutant cancers. The results are presented in FIGS. 15A (BRAF mutant), 15B (NRAS mutant), and 15C (KRAS mutant). In the Figures, the results are presented in % change in BRAF^{V600E} MAF (FIG. 15A), NRAS mutant MAF (FIG. 15B), and KRAS mutant MAF (FIG. 15C), each compared to values measured at patient screening. In the Figures: “CRC” refers to colon cancer; “Mel” refers to melanoma; “Neph” refers to nephroblastoma; “MUO” and “?” each refer to a metastasis of unknown origin; “End” refers to endocrine; “Panc.” refers to pancreatic; “PD” refers to progressive disease; “PR” refers to partial response; and “SD” refers to stable disease.

The results show that there is a more pronounced reduction of allele frequency in PR/SD than PD patients. The results further show the clear effect in BRAF mutant and NRAS mutant patients, whereas the effect is weaker in KRAS mutant patients. The data further shows that ctDNA is a biomarker for progression.

### Example 10

The ctDNA for two of the patients that responded in the clinical trial as shown in FIG. 5 and associated Table 8 was evaluated over the course of treatment period as compared to ctDNA at patient screening. ctDNA was measured by a FoundationACT® blood-based circulating tumor DNA (ctDNA) assay available from Foundation Medicine.

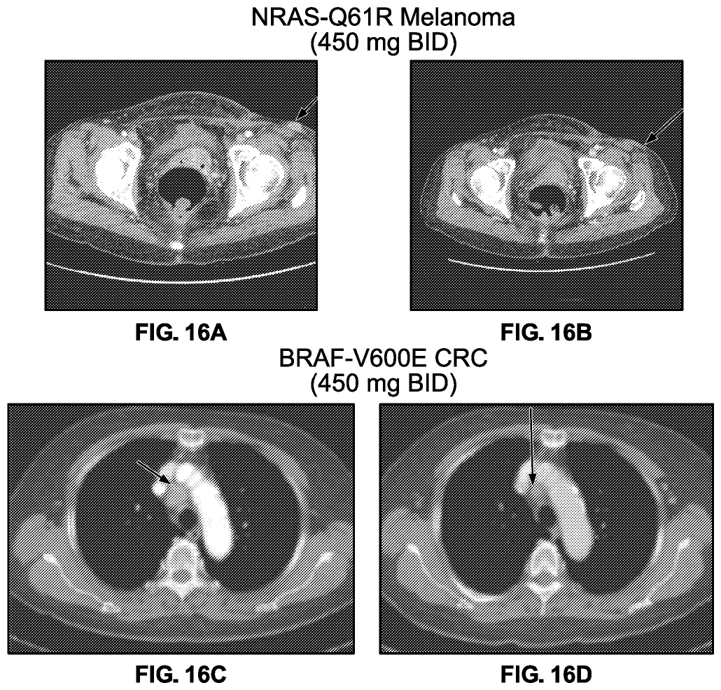

FIG. 16A shows a CT scan of a patient having NRAS^{Q61R} melanoma at the start of treatment and FIG. 16B shows a CT scan of the patient after 8 weeks of treatment with belvarafenib at a dose of 450 mg BID. FIG. 16C shows a CT scan of a patient having BRAF^{V600E} colon cancer at the start of treatment and FIG. 16D shows a CT scan of the patient after 8 weeks of treatment with belvarafenib at a dose of 450 mg BID. The data show that the responder cases correlate with decrease in plasma NRAS^{Q61R} or BRAF^{V600E} ctDNA.

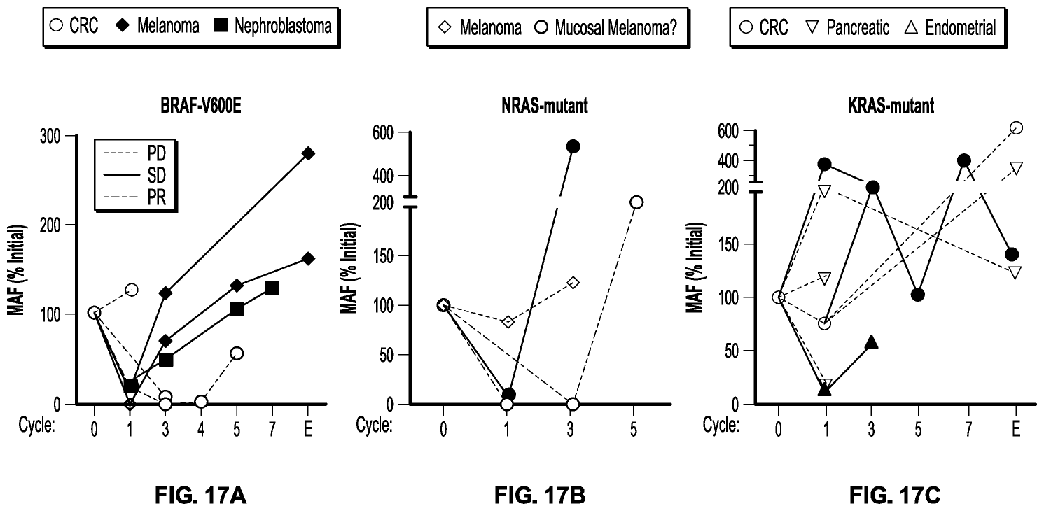

FIGS. 17A to 17C present longitudinal changes in driver mutations in plasma ctDNA levels for BRAF^{V600E} and NRAS^{mut} patients versus KRAS mutant patients. FIG. 17A presents the BRAF^{V600E} MAF ctDNA results versus belvarafenib treatment cycle time for patients having BRAF^{V600E} colon cancer, BRAF^{V600E} melanoma, and BRAF^{V600E} nephroblastoma where the belvarafenib therapy achieved stable disease or partial response, or where the disease progressed. FIG. 17B presents the NRAS mutant MAF ctDNA results versus belvarafenib treatment cycle time for patients having NRAS^{mut} melanoma and NRAS^{mut} mucosal melanoma where the belvarafenib therapy achieved stable disease or partial response. FIG. 17C presents the KRAS mutant MAF ctDNA results versus belvarafenib treatment cycle time for patients having KRAS' colon cancer, KRAS^{mut} pancreatic cancer, KRAS' endometrial cancer where the belvarafenib therapy achieved stable disease, or where the disease progressed. The data show that reduction of allelic frequency in PR/SD is more pronounced than in PD patients.

### Example 11

The in vitro kinase inhibitory selectivity and activity of belvarafenib were evaluated in a kinase panel assay against 189 kinases and a subsequent confirmatory assay against selected kinases by using Z′-Lyte® biochemical assay, Lantha® binding assay and Adapter® assay.

As reported in Table 16 below, belvarafenib showed >90% inhibition of enzymatic activities at 1 μM toward 10 kinases, i.e., BRAF, BRAF^{V599E}, RAF-1 (CRAF) Y340D Y341D, CSF1R (FMS), DDR1, DDR2, EPHA2, EPHA7, EPHA8, and EPHB2. In the confirmatory assay against 6 selected kinases (Table 16), belvarafenib showed potent inhibitory effects on BRAF (IC₅₀=41 nM), BRAF^{V599E} (7 nM), RAF-1 (CRAF), Y340D Y341D (2 nM), CSF1R (FMS) (44 nM), DDR1 (77 nM), and DDR2 (182 nM). The inhibitory effects of belvarafenib on BRAF (41 nM) and BRAF^{V599E} (7 nM) were comparable to those for vemurafenib (38 and 11 nM, respectively), while for RAF-1 (CRAF) Y340D Y341D, belvarafenib (2 nM) was 6 times more potent than vemurafenib (12 nM).

TABLE 16
In vitro Inhibitory Effects of Belvarafenib and Vemurafenib
against 6 Selected Kinases

| | Belvarafenib | Vemurafenib |
|---|---|---|
| Kinase | (IC₅₀ (nM)) | (IC₅₀ (nM)) |
| BRAF | 41 | 38 |
| BRAF^{V599E}* | 7 | 11 |
| RAF-1 Y340D Y341D^{†} | 2 | 12 |
| CSF1R (FMS) | 44 | >10,000 |
| DDR1 | 77 | >10,000 |
| DDR2 | 182 | >10,000 |

*Because of three extra nucleotides found in GC rich exon of BRAF DNA, BRAF^{V600E} mutation was named BRAF^{V599E}.
^{†}Conserved activating mutation at Tyr340/Tyr341 to aspartic acid (RAF-1 Y340D Y341D) increases RAF-1 activity constitutively. Phosphorylation of RAF-1 at Ser338 by Pak (p65Pak) and at Tyr340/Tyr341 by Src family kinases is required for RAF-1 activation.

### Example 12

It is known that despite their efficacy in melanoma with BRAF^{V600} mutations, vemurafenib and dabrafenib are not only ineffective against RAS mutant and RAS/RAF wild-type but also induce ERK activation. For this reason, the MAPK signaling pathway inhibition profiles between belvarafenib versus vemurafenib were investigated using BRAF^{V600E} mutant (SK-MEL-28 and A375) and NRAS mutant (SK-MEL-2 and SK-MEL-30) melanoma cells.

As shown in Table 17 in SK-MEL-28 and A375 BRAF^{V600E} mutant melanoma cells, both belvarafenib and vemurafenib inhibited the phosphorylation of MEK and ERK. On the contrary, in NRAS mutant melanoma cells (SK-MEL-2 and SK-MEL-30), only belvarafenib, but not vemurafenib, showed inhibitory effects on the phosphorylation of MEK and ERK. In vitro cellular IC₅₀ values of belvarafenib for MEK and ERK phosphorylation were 335 and 204 nM in SK-MEL-2, and 388 and 258 nM in SK-MEL-30 cell lines, respectively; the corresponding values for vemurafenib were >10 μM in both SK-MEL-2 and SK-MEL-30 cell lines.

TABLE 17
Inhibition of MEK and ERK Phosphorylation by Belvarafenib and Vemurafenib
in BRAF^{V600E} and NRAS^{Q61R} and NRAS^{Q61K} Mutant Cell Lines

| | | IC₅₀ (nM) | | | |
|---|---|---|---|---|---|
| Cell | | pMEK | | pERK | |
| Lines | Mutation | Belvarafenib | Vermurafenib | Belvarafenib | Vermurafenib |
| SK-MEL-28 | BRAF^{V600E} | 35 | 32 | 155 | 28 |
| A375 | BRAF^{V600E} | 42 | 73 | <1 | 20 |
| SK-MEL-2 | NRAS^{Q61R} | 335 | >10,000 | 204 | >10,000 |
| SK-MEL-30 | NRAS^{Q61K} | 388 | >10,000 | 258 | >10,000 |

### Example 13

The in vitro cell growth inhibitory activity of belvarafenib versus other BRAF inhibitors, vemurafenib and dabrafenib, in melanoma cell lines was assessed both in vemurafenib/dabrafenib-sensitive BRAF^{V600E} mutation harboring SK-MEL-28 and A375 cell lines and in vemurafenib/dabrafenib-resistant melanoma cell lines harboring NRAS mutations, SK-MEL-2 (NRAS^{Q61R}) and SK-MEL-30 (NRAS^{Q61K}). The results are reported in Table 18 and show that belvarafenib potently inhibited not only vemurafenib/dabrafenib-sensitive BRAF mutant melanoma cell lines but also vemurafenib/dabrafenib-resistant NRAS mutant melanoma cell lines. A G150 of 69, 57, 53, and 24 nM were determined for SK-MEL-28, A375, SK-MEL-2, and SK-MEL-30 cell lines, respectively. As expected, vemurafenib and dabrafenib showed inhibitory activity in SK-MEL-28 and A375, but not in SK-MEL-2 and SK-MEL-30 melanoma cell lines.

TABLE 18
In vitro Growth Inhibition (GI₅₀, mean ± SD) of Melanoma Cell
Lines by Belvarafenib versus Vemurafenib and Dabrafenib (n = 3)

| | | GI₅₀ (nM) | | |
|---|---|---|---|---|
| Cell Lines | Mutation | Belvarafenib | Vermurafenib | Dabrafenib |
| SK-MEL-28 | BRAF^{V600E} | 69 ± 6 | 77 ± 17 | <0.1 |
| A375 | BRAF^{V600E} | 57 ± 7 | 75 ± 12 | <0.1 |
| SK-MEL-2 | NRAS^{Q61R} | 53 ± 23 | >10,000 | 204 |
| SK-MEL-30 | NRAS^{Q61K} | 24 ± 6 | >10,000 | 258 |

### Example 14

The in vitro MAPK signaling inhibitory activity of belvarafenib versus other BRAF inhibitors (vemurafenib and dabrafenib) on KRAS mutant cell lines was further investigated in CRC cell lines HCT116 (KRAS^{G13D}) and Lovo (KRAS^{G13D}), and NSCLC cell line, Calu-6 (KRAS^{Q61K}). As shown in Table 19, only belvarafenib, but not vemurafenib and dabrafenib, showed inhibitory effects on the phosphorylation of MEK and ERK in HCT116, Lovo, and Calu-6 cell lines. In vitro cellular IC₅₀ values of belvarafenib for MEK and ERK phosphorylation were 2,698 and 253 nM in HCT116; >10 μM (37% inhibition at 10 μM) and 267 nM in Lovo; and 367 and 590 nM in Calu-6 cell lines, respectively. The corresponding IC₅₀ values for vemurafenib and dabrafenib were >10 μM in HCT116, Lovo, and Calu-6 cell lines.

TABLE 19
Inhibition of MEK and ERK Phosphorylation by Belvarafenib,
Vemurafenib and Dabrafenib in KRAS Mutant CRC and NSCLC
Cell Line

| | | | | IC₅₀ (nM), | |
|---|---|---|---|---|---|
| | | KRAS | | 2 hr treatment | |
| Cancer | Cell Line | mutation | Compound | pMEK | pERK |
| CRC | HCT116 | G13D | Belvarafenib | 2,698 | 253 |
| | | | Vemurafenib | >10,000 | >10,000 |
| | | | Dabrafenib | >10,000 | >10,000 |
| | Lovo | G13D | Belvarafenib | >10,000^{a} | 267 |
| | | | Vemurafenib | >10,000 | >10,000 |
| | | | Dabrafenib | >10,000 | >10,000 |
| NSCLC | Cau-6 | Q61K | Belvarafenib | 367 | 590 |
| | | | Vemurafenib | >10,000 | >10,000 |
| | | | Dabrafenib | >10,000 | >10,000 |

^{a}Belvarafenib inhibited phosphorylation of MEK at 37% at 10 μM.

### Example 15

The in vitro cell growth inhibitory activity of belvarafenib versus other BRAF inhibitors, vemurafenib and dabrafenib, was further investigated in BRAF mutant CRC cell lines: HT-29 and Colo-205 (both BRAF^{V600E}); KRAS mutant CRC cell lines: LS174T (KRAS^{G12D}), LS513 (KRAS^{G12D}), HCT116 (KRAS^{G13D}) and Lovo (KRAS^{G13D}); and KRAS mutant NSCLC cell lines: Calu-6 (KRAS^{Q61K}) and Calu-1 (KRAS^{G12C}). The results are reported in Tables 20 and 21.

While belvarafenib and vemurafenib showed comparable activity on cell growth inhibition in BRAF mutant CRC cell lines, HT-29 and Colo-205 (GI₅₀ range=47-118 nM), dabrafenib showed the most potent cell growth inhibitory effect in those cells with GI₅₀<0.1 nM. Belvarafenib inhibited cell growth in all KRAS mutant CRC cell lines tested in vitro, including: LS174T, LS513, HCT116 and Lovo with G150 values of 258, 62, 177 and 51 nM, respectively (Table 20). Activity of belvarafenib on cell growth inhibition of KRAS mutant NSCLC cell lines was also observed in Calu-6 and Calu-1 (GI₅₀ of 179 and 749 nM, respectively) (Table 21). Dabrafenib also showed in vitro cell growth inhibition in Lovo (KRAS mutant, CRC) cell line (GI₅₀=214 nM), and Calu-6 and Calu-1 (KRAS mutant, NSCLC) cell lines (GI₅₀ of 618 and 904 nM, respectively). The activities of dabrafenib, however, were about 3 to 4-fold weaker than belvarafenib, except in Calu-1 cells. Vemurafenib showed no activity on the inhibition of growth in KRAS mutant cells.

TABLE 20
In vitro Cell Growth Inhibition of BRAF or KRAS Mutant
Colorectal Cancer (CRC) Cell Lines by Belvarafenib versus
Vemurafenib and Dabrafenib

| | | GI₅₀ (nM) | | |
|---|---|---|---|---|
| Cell Line | Mutation | Belvarafenib | Vemurafenib | Dabrafenib |
| HT-29 | BRAF^{V600E} | 47 | 110 | <0.1 |
| Colo-205 | BRAF^{V600E} | 118 | 65 | <0.1 |
| LS174T | KRAS^{G12D} | 258 | >10,000 | >10,000 |
| LS513 | KRAS^{G12D} | 62 | >10,000 | 3,059 |
| HCT116 | KRAS^{G13D} | 177 ± 103 | >10,000 | >10,000 |
| Lovo | KRAS^{G13D} | 51 ± 19 | 4,025 ± 11 | 214 ± 61 |

TABLE 21
In vitro Cell Growth Inhibition of KRAS Mutant Non-Small
Cell Lung Cancer (NSCLC) Cell Lines by Belvarafenib versus
Vemurafenib and Dabrafenib

| | | GI₅₀ (nM) | | |
|---|---|---|---|---|
| Cell Line | Mutation | Belvarafenib | Vemurafenib | Dabrafenib |
| Calu-6 | KRAS^{Q61K} | 179 ± 103 | >10,000 | 618 ± 252 |
| Calu-1 | KRAS^{G12C} | 749 | 7,476 | 904 |

### Example 16

The in vitro cell growth inhibitory activity of belvarafenib versus other BRAF inhibitors, vemurafenib and dabrafenib, on BRAF or KRAS mutant cell lines was further investigated in BRAF mutant thyroid cell lines: SNU790, FRO, B-CPAP, NPA, 8505C, ARO (all BRAF^{V600E}) and SNU80 (BRAF^{G468R}); and KRAS mutant thyroid cancer cell line, CAL-62 (KRAS^{G12R}). The results are reported in Table 22.

Belvarafenib and dabrafenib showed activity on cell growth inhibition in all 7 BRAF mutant thyroid cancer cell lines (GI₅₀, <1 μM). Vemurafenib showed cell growth inhibitory effect in SNU790, B-CPAP and NPA, BRAF mutant thyroid cancer cell lines, with G150 values<1 μM. Additionally, only belvarafenib, but not vemurafenib or dabrafenib, showed activity on cell growth inhibition in CAL-62 (KRAS^{G12R}) thyroid cancer cells, with G150 value of 479 nM.

TABLE 22
In vitro Cell Growth Inhibition of BRAF or KRAS Mutant Thyroid
Cancer Cell Lines by Belvarafenib versus Vemurafenib and Dabrafenib

| | | GI₅₀ (nM) | | |
|---|---|---|---|---|
| Cell Line | Mutation | Belvarafenib | Vemurafenib | Dabrafenib |
| SNU7890 | BRAF^{V600E} | 27 | 190 | 0.1 |
| FRO | BRAF^{V600E} | 30 | 1,763 | 23 |
| B-CPAP | BRAF^{V600E} | 43 | 31 | <0.1 |
| NPA | BRAF^{V600E} | 45 | 44 | <0.1 |
| 8505C | BRAF^{V600E} | 345 | 3,444 | 184 |
| ARO | BRAF^{V600E} | 719 | 5,260 | 548 |
| SNU80 | BRAF^{G468R} | 163 | 2,848 | 51 |
| CAL-62 | KRAS^{G12R} | 479 | >10,000 | >10,000 |

### Example 17

In vivo antitumor activity of belvarafenib was investigated in NRAS^{G13D} mutant K1735 syngeneic mouse melanoma model. Seven animals per group were treated with vehicle (control) and with belvarafenib at a dose of 7.5 or 15 mg/kg once daily via oral gavage. As shown in Table 23, on day 22 the maximum inhibition rate (mIR) for belvarafenib was 48.2% at 7.5 mg/kg and 54.7% at 15 mg/kg. Inhibition rate (%)=(1−mean relative tumor weight in treated group/mean relative tumor weight in control group)×100.

TABLE 23
In vivo Antitumor Activity of Belvarafenib Administered
for 3 weeks in NRAS^{G13D} mutant K1735 syngeneic mouse
melanoma model

| | 7.5 mg/kg | 15 mg/kg |
|---|---|---|
| Maximum inhibition rate (%) | 48.2 | 54.7 |
| Day for maximum inhibition | 22 | 22 |
| Maximum weight loss (%) | 2.8 | 2.2 |

### Example 18

In vivo antitumor activity of belvarafenib was investigated in mice model xenografted with SK-MEL-30 human melanoma cell line harboring NRAS^{Q61K} mutation. Five animals per group were treated with vehicle (control) and belvarafenib at a dose of 10 or 30 mg/kg once daily via oral gavage up to day 14.

As shown in Table 24, oral administration of belvarafenib resulted in dose-dependent antitumor activity with 70.3% (on day 15) and 80.0% (on day 15) of the maximum inhibition rate at 10 and 30 mg/kg q.d., respectively. Treatment with belvarafenib was well tolerated without body weight loss. Clinical sign of hair growth on the back was observed.

TABLE 24
In vivo Antitumor Activities of Belvarafenib Administered
Orally for 14 days in Mice Xenografted with SK-MEL-30
Melanoma Cancer Cell Line

| | 10 mg/kg | 30 mg/kg |
|---|---|---|
| Maximum inhibition rate (%) | 70.3 | 80.0 |
| Day for maximum inhibition | 15 | 15 |
| Maximum weight loss (%) | — | — |

### Example 19

In vivo antitumor activity of belvarafenib was investigated in a second experiment in a mice model xenografted with SK-MEL-30 human melanoma cell line harboring NRAS^{Q61K} mutation. Five animals per group were treated with vehicle (control) and belvarafenib at a dose of 10 or 30 mg/kg once daily via oral gavage up to day 21.

As shown in Table 25, oral administration of belvarafenib resulted in dose-dependent antitumor activity with 36.7% (on day 21) and 74.6% (on day 21) of the maximum inhibition rate at 10 and 30 mg/kg q.d., respectively.

TABLE 25
In vivo Antitumor Activities of Belvarafenib Administered
Orally for 21 days in Mice Xenografted with SK-MEL-30
Melanoma Cancer Cell Line

| | 10 mg/kg | 30 mg/kg |
|---|---|---|
| Maximum inhibition rate (%) | 36.7 | 74.6 |
| Day for maximum inhibition | 21 | 21 |
| Maximum weight loss (%) | — | — |

### Example 20

In vivo antitumor activity of belvarafenib was investigated in an experiment in a mice model xenografted with a HT-29 CRC cell line harboring BRAF^{V600E} mutation. Five animals per group were treated with vehicle (control) and belvarafenib at a dose of 30 mg/kg once daily via oral gavage up to day 21.

As shown in Table 26, oral administration of belvarafenib resulted in antitumor activity with 59.8% (on day 22) of the maximum inhibition rate at 30 mg/kg.

TABLE 26
In vivo Antitumor Activities of Belvarafenib Administered
Orally for 21 days in Mice Xenografted with a HT-29 CRC
cell line harboring BRAF^{V600E} mutation

| | 30 mg/kg |
|---|---|
| Maximum inhibition rate (%) | 59.8 |
| Day for maximum inhibition | 21 |
| Maximum weight loss (%) | 2.4 |

### Example 21

In vivo antitumor activity of belvarafenib was investigated in an experiment in a mice model xenografted within a Calu-6 NSCLC cell line harboring KRAS^{Q61K} mutation. Five animals per group were treated with vehicle (control) and belvarafenib at a dose of 3, 10, or 30 mg/kg once daily via oral gavage for 17 days.

As shown in Table 27, oral administration of belvarafenib resulted in dose-dependent antitumor activity with 53.5% (on day 18), 79.3% (on day 15) and 86.3% (on day 12) of the maximum inhibition rate at 3, 10, and 30 mg/kg q.d., respectively.

TABLE 27
In vivo Antitumor Activity of Belvarafenib
Administered Orally for 17 Days in Mice Xenografted
with Calu-6 Non-Small Cell Lung Cancer Cell Line

| | 3 | 10 | 30 |
|---|---|---|---|
| | mg/kg | mg/kg | mg/kg |
| Maximum inhibition rate (%) | 53.5 | 79.3 | 86.3 |
| Day for maximum inhibition | 18 | 15 | 12 |
| Maximum weight loss (%) | 2.5 | 3.0 | 1.1 |

### Example 22

A phase Ib, multicenter study will be done to evaluate the safety, pharmacokinetics, and activity of belvarafenib as a single agent in patients with NRAS-mutant metastatic or unresectable locally advanced cutaneous melanoma who have received up to two lines of systemic anti-cancer therapy that included anti-PD 1/PD-L1 therapy.

This study will enroll patients with measurable disease (according to RECIST v1.1), advanced melanoma as defined by the American Joint Committee on Cancer, 8th revised edition (Amin et al. 2017), harboring an NRAS-activating mutation.

The patients will have Documentation of NRAS mutation-positive status in melanoma tumor tissue (archival or newly obtained), as determined by the local laboratory within 5 years prior to screening, through use of a clinical mutation test approved by the local health authority (e.g., U.S. Food and Drug Administration [FDA] approved test, College of American Pathologists, CE-marked [European conformity] in vitro diagnostic in E.U. countries, or equivalent). NRAS mutation-positive status is defined as a mutation occurring in NRAS gene codons 12, 13 of exon 2, and codon 61 of exon 3.

Up to 15 patients will be enrolled and will receive 300 mg or 400 mg belvarafenib twice a day (BID) in tablet form on Days 1-28 of each 28-day cycle. Belvarafenib will be administered within 30 minutes of a meal.

To characterize the PK profile and immunogenic response of study treatment, blood samples will be taken at various timepoints before and after dosing. PK parameters will be derived from the plasma concentrations of belvarafenib versus time from dose using noncompartmental methods, when appropriate for Cycle 1, Day 1 and steady-state: Cₘₐₓ, tₘₐₓ, area under the concentration-time curve (AUC) from nominal time 0 to time t (AUC₀₋ₜ). Furthermore, plasma concentrations of belvarafenib will be reported as individual values and summarized when appropriate and as data allow. Individual and mean belvarafenib concentrations will be plotted by treatment arm and day. Belvarafenib concentration data may be pooled with data from other studies using an established population PK model to derive PK parameters such as clearance, volume of distribution, and AUC, as warranted by the data. Potential correlations of relevant PK parameters with dose, safety, efficacy, or biomarker outcomes may be explored.

A minimum of 5 patients will be required to undergo three serial biopsies at the following timepoints: at screening (after other eligibility criteria have been fulfilled), 6 weeks after initiation of study treatment, and at the time of disease progression. Additional biopsies from these patients may be collected at the investigator's discretion.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1-18. (canceled)

19. A method for treating a cancer in a human subject, comprising administering an effective amount of belvarafenib to the human subject, wherein the cancer has at least one mutation selected from a BRAF mutation, a KRAS mutation, and a NRAS mutation, wherein the cancer has at least one mutation selected from a BRAF^{G468R} mutation, a BRAF^{V599E} mutation, a KRAS^{G12R} mutation, a KRAS^{Q61K} mutation, a KRAS^{Q61L} mutation, a NRAS^{G13D} mutation, and a NRAS^{Q61L} mutation.

20. The method of claim 19, wherein the cancer comprises melanoma, nephroblastoma, GIST, CRC, NSCLC, sarcoma, gallbladder cancer, bladder cancer, thyroid cancer, and any combinations thereof.

21. The method of claim 19, wherein the cancer is selected from: (1) thyroid cancer carrying a BRAF^{G468R} mutation, thyroid cancer carrying a KRAS^{G12R} mutation, and a combination thereof; (2) NSCLC carrying a KRAS^{Q61K} mutation; (3) CRC carrying a KRAS^{Q61L} mutation; (4) melanoma carrying a NRAS^{G13D} mutation, melanoma carrying a NRAS^{Q61L} mutation, and a combination thereof; and (5) combinations thereof.

22. The method of claim 19, wherein from 200 mg per day of belvarafenib to 1300 mg per day of belvarafenib is administered to the human subject.

23. The method of claim 22, wherein 450 mg BID of belvarafenib per day is administered to the subject.

24. The method of claim 19, wherein said method for treating cancer is characterized by the absence of the development of squamous cell carcinoma in the human subject.

25. The method of claim 19, wherein:
(1) the cancer is melanoma; and
(2) prior to said belvarafenib treatment, the subject experienced disease progression after treatment with immunotherapy, BRAF^{V600E} therapy, or a combination of immunotherapy and BRAF^{V600E} therapy.

26. A method for treating a cancer in a human subject, comprising administering an effective amount of belvarafenib to the human subject, wherein the cancer has at least one mutation selected from a BRAF mutation, a KRAS mutation, and a NRAS mutation, and wherein the cancer is selected from thyroid cancer and non-small cell lung cancer.

27. The method of claim 26, wherein the at least one mutation is selected from a BRAF^{V600E} mutation, a BRAF^{G468R} mutation, a BRAF^{V599E} mutation, a KRAS^{G12C} mutation, a KRAS^{G12D} mutation, a KRAS^{G13D} mutation, a KRAS^{G12V} mutation, a KRAS^{G12R} mutation, a KRAS^{Q61H} mutation, a NRAS^{G12C} mutation, a NRAS^{G12D} mutation, a NRAS^{G13D} mutation, a NRAS^{Q61K} mutation, a NRAS^{Q61L} mutation, and a NRAS^{Q61R} mutation.

28. The method of claim 26, wherein the cancer is selected from: (1) thyroid cancer carrying a BRAF^{V600E} mutation, thyroid cancer carrying a BRAF^{G468R} mutation, thyroid cancer carrying a KRAS^{G12R} mutation, and combinations thereof; and (2) NSCLC carrying a KRAS^{G12C} mutation, NSCLC carrying a KRAS^{Q61K} mutation, NSCLC carrying a NRAS^{Q61K} mutation, and combinations thereof; and (3) combinations thereof.

29. The method of claim 26, wherein from 200 mg per day of belvarafenib to 1300 mg per day of belvarafenib is administered to the human subject.

30. The method of claim 29, wherein 450 mg BID of belvarafenib is administered to the subject.

31. The method of claim 26, wherein said method for treating cancer is characterized by the absence of the development of squamous cell carcinoma in the human subject.

32. A method for treating a cancer in a human subject, comprising administering an effective amount of belvarafenib to the human subject, wherein the cancer is selected from the group consisting of:
(1) melanoma carrying a BRAF^{G468R} mutation, a BRAF^{V599E} mutation, a KRAS^{G12C} mutation, a KRAS^{G12C} mutation, a KRAS^{G12D} mutation, a KRAS^{G13D} mutation, a KRAS^{G12V} mutation, a KRAS^{G12R} mutation, a KRAS^{Q61H} mutation, a KRAS^{Q61K} mutation, a KRAS^{Q61L} mutation, a NRAS^{G13D} mutation, a NRAS^{Q61L} mutation, or any combination thereof;
(2) GIST carrying a BRAF^{G468R} mutation, a BRAF^{V599E} mutation, a KRAS^{G12C} mutation, a KRAS^{G12D} mutation, a KRAS^{G13D} mutation, a KRAS^{G12V} mutation, a KRAS^{G12R} mutation, a KRAS^{Q61H} mutation, a KRAS^{Q61K} mutation, a KRAS^{Q61L} mutation, a NRAS^{G12C} mutation, a NRAS^{G12D} mutation, a NRAS^{G13D} mutation, a NRAS^{Q61K} mutation, a NRAS^{Q61L} mutation, a NRAS^{Q61R} mutation, or any combination thereof;
(3) CRC carrying a BRAF^{G468R} mutation, a BRAF^{V599E} mutation, a KRAS^{G12D} mutation, a KRAS^{G12V} mutation, a KRAS^{G12R} mutation, a KRAS^{Q61K} mutation, a KRAS^{Q61L} mutation, a NRAS^{G12C} mutation, a NRAS^{G12D} mutation, a NRAS^{G13D} mutation, a NRAS^{Q61K} mutation, a NRAS^{Q61L} mutation, a NRAS^{Q61R} mutation, or any combination thereof;
(4) nephroblastoma carrying a BRAF^{G468R} mutation, a BRAF^{V599E} mutation, a KRAS^{G12C} mutation, a KRAS^{G12D} mutation, a KRAS^{G13D} mutation, a KRAS^{G12V} mutation, a KRAS^{G12R} mutation, a KRAS^{Q61H} mutation, a KRAS^{Q61K} mutation, a KRAS^{Q61L} mutation, a NRAS^{G12C} mutation, a NRAS^{G12D} mutation, a NRAS^{G13D} mutation, a NRAS^{Q61K} mutation, a NRAS^{Q61L} mutation, a NRAS^{Q61R} mutation, or any combination thereof;
(5) bladder cancer carrying a BRAF^{V600E} mutation, a BRAF^{G468R} mutation, a BRAF^{V599E} mutation, a KRAS^{G12C} mutation, a KRAS^{G13D} mutation, a KRAS^{G12R} mutation, a KRAS^{Q61H} mutation, a KRAS^{Q61K} mutation, a KRAS^{Q61L} mutation, a NRAS^{G12C} mutation, a NRAS^{G12D} mutation, a NRAS^{G13D} mutation, a NRAS^{Q61K} mutation, a NRAS^{Q61L} mutation, a NRAS^{Q61R} mutation, or any combination thereof;
(6) gallbladder cancer carrying a BRAF^{V600E} mutation, a BRAF^{G468R} mutation, a BRAF^{V599E} mutation, a KRAS^{G12C} mutation, a KRAS^{G13D} mutation, a KRAS^{G12V} mutation, a KRAS^{G12R} mutation, a KRAS^{Q61H} mutation, a KRAS^{Q61K} mutation, a KRAS^{Q61L} mutation, a NRAS^{G12C} mutation, a NRAS^{G12D} mutation, a NRAS^{G13D} mutation, a NRAS^{Q61K} mutation, a NRAS^{Q61L} mutation, a NRAS^{Q61R} mutation, or any combination thereof;
(7) sarcoma carrying a BRAF^{V600E} mutation, a BRAF^{G468R} mutation, a BRAF^{V599E} mutation, a KRAS^{G12C} mutation, a KRAS^{G12D} mutation, a KRAS^{G13D} mutation, a KRAS^{G12R} mutation, a KRAS^{Q61H} mutation, a KRAS^{Q61K} mutation, a KRAS^{Q61L} mutation, a NRAS^{G12C} mutation, a NRAS^{G12D} mutation, a NRAS^{G13D} mutation, a NRAS^{Q61K} mutation, a NRAS^{Q61L} mutation, a NRAS^{Q61R} mutation, or any combination thereof; and
(8) combinations thereof.

33. The method of claim 32, wherein the cancer is: (1) melanoma carrying a KRAS^{G12V} mutation, a NRAS^{G13D} mutation, a NRAS^{Q61L} mutation, and combinations thereof; (2) CRC carrying a KRAS^{G12D} mutation, a KRAS^{Q61L} mutation, and combinations thereof; and (3) combinations thereof.

34. The method of claim 33, wherein the cancer is selected from melanoma and GIST.

35. The method of claim 32, wherein from 200 mg per day of belvarafenib to 1300 mg per day of belvarafenib is administered to the human subject.

36. The method of claim 35, wherein 450 mg BID of belvarafenib is administered to the subject.

37. The method of claim 32, wherein said method for treating cancer is characterized by the absence of the development of squamous cell carcinoma in the human subject.

38. The method of claim 32, wherein:
(1) the cancer is melanoma; and
(2) prior to said belvarafenib treatment, the subject experienced disease progression after treatment with immunotherapy, BRAF^{V600E} therapy, or a combination of immunotherapy and BRAF^{V600E} therapy.
